SEQUENCE LISTING (7 sequences)

SEQ ID NO: 1 - protein, 17 aa, Mycobacterium tuberculosis
WDATATELNNALQNLAR

SEQ ID NO: 2 - protein, 19 aa, Mycobacterium tuberculosis
LAAAWGGSGSEAYQGVQQK

SEQ ID NO: 3 - protein, 18 aa, Mycobacterium tuberculosis
TQIDQVESTAGSLQGQWR

SEQ ID NO: 4 - protein, 15 aa, Mycobacterium tuberculosis
ADEEQQQALSSQMGF

SEQ ID NO: 5 - protein, 15 aa, Mycobacterium tuberculosis
TDAATLAQEAGNFER

SEQ ID NO: 6 - protein, 13 aa, Mycobacterium tuberculosis
GAAGTAAQAAVVR

SEQ ID NO: 7 - protein, 8 aa, Mycobacterium tuberculosis
QAGVQYSR

(19) United States
(12) Patent Application Publication
(10) Pub. No.: US 2015/0260715 A1
(43) Pub. Date: Sep. 17, 2015

(54) RAPID DETECTION AND QUANTITATION OF PATHOGEN-SPECIFIC BIOMARKERS USING NANOPOROUS DUAL- OR MULTI-LAYER SILICA FILMS

(71) Applicant: The Methodist Hospital Research Institute, Houston, TX (US)

(72) Inventors: Ye HU, Houston, TX (US); Xin MA, Pearland, TX (US); Hung-Jen WU, College Station, TX (US); Jia FAN, Houston, TX (US); Tong SUN, Houston, TX (US)

(21) Appl. No.: 14/720,715

(22) Filed: May 22, 2015

Related U.S. Application Data
(63) Continuation of application No. PCT/US2013/072416, filed on Nov. 27, 2013.
(60) Provisional application No. 61/732,266, filed on Nov. 30, 2012.

(51) Int. Cl. G01N 33/569; G01N 33/552

(52) U.S. Cl. CPC G01N 33/5695; G01N 33/552; G01N 2469/10; G01N 2333/35

(57) ABSTRACT

Improved methods for detecting active tuberculosis are disclosed. A method comprises enriching at least one M. tuberculosis-specific biomolecule from a sample by contacting the sample with a nanoporous film; and detecting the presence of the M. tuberculosis-specific biomolecule or fragment(s) thereof. The method may further comprise digesting the enriched M. tuberculosis-specific biomolecule with an enzyme to produce a digestion product comprising at least one fragment of the M. tuberculosis-specific biomolecule. Improved sensitivity and speed achieved.

### CROSS-REFERENCE TO RELATED APPLICATIONS

The present application claims priority to PCT Intl. Pat. Appl. No. PCT/US2013/072416; filed Nov. 27, 2013 (pending; Atty. Dkt. No. 37182.163), which claims the benefit of U.S. Prov. Pat. Appl. No. 61/732,266, filed Nov. 30, 2012 (expired; Atty. Dkt. No. 37182.162), the contents of each of which is specifically incorporated herein in its entirety by express reference thereto.

### STATEMENT REGARDING FEDERALLY SPONSORED RESEARCH OR DEVELOPMENT

This invention was made with government support under Grant No. W81XWH-11-2-0168 awarded by the United States Department of Defense, and Grant No. U54-CA-151668 awarded by the National Institutes of Health. The government has certain rights in the invention.

### NAMES OF THE PARTIES TO A JOINT RESEARCH AGREEMENT

Not Applicable.

### BACKGROUND OF THE INVENTION

1. Field of the Invention

The present invention relates to the fields of molecular biology and medicine. In particular, the invention provides methods and compositions for the detection of tuberculosis in a sample. In illustrative embodiments, the invention provides a label-free, rapid (˜one-hour), and cost-effective high-throughput diagnostic assay that detects both pulmonary and extra-pulmonary active tubercular disease.

2. Description of Related Art

With the increase of international travel and immigration, control of infectious disease such as tuberculosis (TB) is more challenging than any time in the history of public health. Annual worldwide statistics indicate nine million new cases and 1.5 million deaths from tuberculosis (TB), an airborne infectious disease caused by Mycobacterium tuberculosis (MTB), establishing TB as a continued significant public health challenge. A major contributing factor against global TB control has been unfavorably influenced by the human immunodeficiency virus (HIV) epidemic and the emergence of tenacious multidrug and extensively drug-resistant TB (M/XDR-TB). Identification of TB in pediatric patients and the highly vulnerable human-immunodeficiency virus (HIV) population is even more challenging with the paucibacillary nature of the disease, non-specific symptoms, and difficulties in obtaining clinically relevant specimens.

Traditional TB testing requires subjects to receive an under-the-skin injection of a harmless protein produced by Mycobacterium tuberculosis. Two or three days later, the subject is asked to return for a “reading,” during which the injection site is evaluated. If a person has ever been infected by TB or is currently battling a TB infection, the injection site will appear red and irritated—a positive reading.

M. tuberculosis culture testing (MTCT) remains the “gold standard” for the diagnosis of active TB disease, as well as the identification of drug-resistance. Unfortunately, this method generally requires 6 to 8 weeks to complete (Dunlap et al., 2000; Scarpellini et al., 2004). In addition to the significant delay in receiving results, conventional TB tests are unable to detect some types of active TB disease, such as tuberculous meningitis, which does not actively shed bacteria. Sputum smear microscopy, the primary means of tuberculosis diagnosis in most parts of the world for more than a century, requires well-trained personnel to be performed correctly, and its sensitivity (35-80%) is significantly lower than that of mycobacterial culture. The method has a number of drawbacks, including low sensitivity (especially in HIV-positive individuals and children) and an inability to determine drug-resistance. Because conventional diagnosis of drug resistant TB relies on bacterial culture and drug susceptibility testing, both slow and cumbersome processes, during that time patients may be inappropriately treated, drug-resistant strains may continue to spread, and resistance may become amplified. The method is also unable to differentiate between drug-sensitive and drug-resistant tuberculosis strains.

To determine whether TB bacteria are resistant to antibiotic drugs, current technology require a separate test that takes 3-6 weeks to complete. Rapid and reliable diagnostic tests for active tubercular disease are thus highly desirable to minimize the morbidity and mortality of this airborne infectious disease.

ESAT-6 and CFP-10 as Biomarkers of Tuberculosis

ESAT-6 (early secretory antigenic target protein) and CFP-10 (culture filtrate protein 10) are exclusively secreted by several pathogenic mycobacterial species, including M. tuberculosis, and non-tuberculosis species (NTM, M. kansasii, M. szulgai, M. marinum, and M. riyadhense), and is consistently missing from all versions of attenuated vaccine strains (Bacillus Calmette-Guérin, BCG) and other mycobacterial species (van Ingen et al., 2009). Thus, ESAT-6 and CFP-10 are considered excellent biomarkers for TB diagnosis.

The interferon-gamma release assays (IGRAs), immunodiagnostic assays, were developed and commercialized to detect ESAT-6-immunized T-cells in whole blood (Doherty et al., 2002). Although the IGRAs are sensitive to those who have been administered the BCG vaccine, this assay still cannot distinguish between active TB disease and remote latent TB infection (LTBI), due to the immunologic response from long-lived human memory T cells (Wu-Hsieh et al., 2001). Since actively replicating M. tuberculosis strains within the human body release ESAT-6, which triggers chronic inflammation and an immune response, ESAT-6 could be detected in a patient's bodily fluids, including sputum (pulmonary TB), serum, cerebrospinal fluids (tuberculous meningitis), and pleural effusion (tuberculous pleuritis) (Kashyap et al., 2009; Sang et al., 2012; Hoff et al., 2007; Ravn et al., 2005).

Nucleic acid and nanoparticle approaches have been developed in recent years. However, most nucleic acid-based approaches require polymerase chain reaction (PCR) methodology for amplifying nucleic acids specific to the TB bacteria, one in particular uses a nested protocol that targets the heat shock protein 65 gene (hsp65). Most nanoparticle-based approaches merely use nanoparticles to detect antigens that are already captured (Chun, 2009; Torres-Chavolla and Alocilia, 2011). Spherical gold nanoparticles have also been employed for rapid detection of MT-specific DNA using non-PCR based protocols (Hussain et al., 2013; Tsai et al., 2013).

A need remains, however, for faster, more sensitive, and more specific methods of TB detection, including assays that are suitable for detecting the bacterium in patient-derived, biological specimens. Unique and more varied assays are needed, including peptide- and protein-based approaches that do not rely on nanoparticle-based methodologies.

### BRIEF SUMMARY OF THE INVENTION

The present invention overcomes these and other limitations inherent in the prior art by providing inventive diagnostic compositions for use in the preparation of medicaments, and in methods for the detection, diagnosis, treatment, and/or amelioration of one or more symptoms of mammalian disease. In particular, the invention provides novel, non-obvious, and useful compositions that are suitable for the detection of the causal agent of tubercular infection in mammals, and in particular, for the detection of symptoms of M. tuberculosis (MTB) cells that express an MTB-specific biomarker, such as ESAT-6 or CFP-10 proteins, peptides, or one or more proteolytic fragments derived therefrom.

Importantly, the present invention provides label-free, highly reproducible diagnostic tests that are cost-effective, and capable of identifying active TB disease including the more dangerous multi-drug-resistant tuberculosis. The disclosed methods provide rapid diagnosis of TB infections (typically within one hour from sample collection to diagnosis), and importantly, can be used to distinguish between active TB disease and latent TB infection.

The present invention utilizes nanoporous silica chips that are constructed with a dual-layered film, engineered with properties that facilitate on-chip fractionation and digestion of samples, exclusion of the abundant proteins that normally obscure the detection of the target molecules and selective capture of the rare biomarkers from a biological sample. The diagnostic chips described herein are useful in selectively purifying low-molecular-weight (LMW) TB biomarkers, and facilitating the highly sensitive detection and quantification of such biomarkers by an analytical method such as mass spectrometry (MS).

Use of such diagnostic chips to selectively purify LMW TB biomarkers within the nanomolar range facilitates a reproducible, cost-effective, and high-throughput (e.g., ˜150 sample wells in a four-inch-size chip) platform, which permits highly sensitive detection and quantification of biomarkers of interest by MS.

In a first embodiment, the invention provides a method of identifying at least one pathogen-specific protein or peptide from a sample. In an overall and general sense, the method generally involves contacting the sample with a nanoporous film; and detecting the presence of the pathogen-specific protein or peptide, or one or more proteolytic fragment(s) thereof

In certain embodiments, the pathogen-specific protein or peptide is specific for M. tuberculosis, and may include a contiguous amino acid sequence from an early secretory antigenic target protein (ESAT-6), a culture filtrate protein 10 (CFP-10), or one or more proteolytic fragment(s) thereof.

In certain embodiments, the pathogen-specific protein or peptide comprises, consists essentially of, or alternatively, consists of at least 8, at least 9, or at least 10 or more contiguous amino acids from any one of SEQ ID NO:1, SEQ ID NO:2, SEQ ID NO:3, SEQ ID NO:4, SEQ ID NO:5, SEQ ID NO:6, and SEQ ID NO:7.

Alternatively, the pathogen-specific protein or peptide may comprise, consist essentially of, or alternatively, consist of at least 11, at least 12, or at least 13 contiguous amino acids from any one of SEQ ID NO:1, SEQ ID NO:2, SEQ ID NO:3, SEQ ID NO:4, SEQ ID NO:5, or SEQ ID NO:6.

In particular embodiments, the pathogen-specific protein or peptide may comprise, consist essentially of, or alternatively, consist of at least 14, at least 15, or at least 16 contiguous amino acids from any one of SEQ ID NO:1, SEQ ID NO:2, SEQ ID NO:3, SEQ ID NO:4, or SEQ ID NO:5. And in other embodiments, the pathogen-specific protein or peptide may comprise, consist essentially of, or alternatively, consist of at least 17, or at least 18 contiguous amino acids from any one of SEQ ID NO:1, SEQ ID NO:2, SEQ ID NO:3, or SEQ ID NO:4.

Preferably, the sample is a biological sample obtained from a mammal, and more particularly is a biological sample that contains sputum, pleural effusion, cerebrospinal fluid, urine, serum, plasma, and/or whole blood obtained from a human.

In some applications, the sample may be contacted with the film neat (i.e., undiluted), or alternatively, the at least one protein or peptide within the sample may be concentrated prior to contact with the nanoporous film, using a suitable method such as salt precipitation or such like.

Preferably, the nanoporous film comprises a plurality of pores, substantially having the same average diameter, into which the pathogen-specific protein or peptide is absorbed. The nanoporous film may comprise a first layer of silica film that contains a plurality of pores having an average diameter of about three to about ten nm, or alternatively, may contain a first layer of silica film that contains a plurality of pores having an average diameter of about six to about eight nm.

In certain embodiments, the nanoporous film may further optionally include a second layer of silica film deposited upon the first layer, to form a dual-layer film. In such applications, preferably, the first layer of silica film contains a plurality of pores having a first average diameter, and the second layer of silica film contains a plurality of pores having a second average diameter that is distinct from that of the first layer.

In certain commercial applications, the second layer of silica film may contain a plurality of pores having a first average diameter that is larger than that of the plurality of pores in the first layer.

The method may also further optionally include washing the nanoporous film after contacting the film with the sample, and/or digesting the sample containing the pathogen-specific protein or peptide with a protease or a peptidase to produce one or more proteolytic fragment(s) of the pathogen-specific protein or peptide.

For detection of MTB-specific peptides, the protease is preferably trypsin. The proteolysis of the sample may be performed prior to analysis, or alternatively, upon or within the nanoporous film itself during analysis.

The method may further optionally include isolating the one or more proteolytic fragment(s) from the nanoporous film with a suitable elution buffer.

The presence of the pathogen-specific protein or peptide, or the one or more proteolytic fragment(s) thereof is preferably detected by identifying at least one mass fingerprint of the protein, the peptide, or the proteolytic fragment(s) thereof by MS.

In the analysis of ESAT-6 or CFP-10, the at least one mass fingerprint is identified at about 1895-1910 Da ([M+H]⁺) or about 2003-2005 Da ([M+H]⁺). or alternatively at about 1900.9511 Da ([M+H]⁺) or 1907.9246 Da ([M+H]⁺) or alternatively at about 2003.9781 Da ([M+H]⁺) about 1668.7170 Da ([M+H]⁺) about 1593.7503 Da ([M+H]⁺) about 1142.6276 Da ([M+H]⁺) or about 908.4584 Da ([M+H]⁺) The at least one mass fingerprint may be detected by matrix-assisted laser desorption/ionization time-of-flight mass spectrometry (MALDI-TOF MS).

In other aspects, the invention provides a method that generally includes the steps of a) enriching at least one target protein from a sample by contacting the sample with a nanoporous film under conditions to absorb the target protein to the film, and subsequently washing the nanoporous film to remove extraneous material; b) digesting the enriched target protein on the nanoporous film to produce at least one digestion product comprising at least one proteolytic fragment thereof; and c) detecting the presence of the at least one proteolytic fragment of the target protein. In the practice of the method, at least two different target proteins, either or both of which is specific for a particular pathogen, may be from a single sample, or alternatively, may be from multiple samples.

In certain embodiments, the nanoporous film may include a silica film that contains a plurality of pores having an average diameter of about 3 to about 10 nm, and as noted above, may also include dual- or multi-layer silica films each having substantially same, or substantially different average pore sizes contained therewith to provide differential sorting of the proteins or peptides in the sample based upon size of the pores of each layer.

In another embodiment, the invention provides a method that generally includes the steps of (a) enriching at least one ESAT-6-, CFP-10-, or IF-10-specific protein or peptide from a sample containing a first mammalian bodily fluid, by contacting the sample with a nanoporous film and washing the nanoporous film, wherein the nanoporous film comprises a plurality of pores having an average diameter of about 3 to about 10 nm; (b) digesting the enriched ESAT-6-, CFP-10-, or IF-10-specific protein or peptide in the nanoporous film with at least one protease to produce a digestion product comprising at least one proteolytic fragment of an ESAT-6-, CFP-10-, or IF-10-specific protein or peptide; (c) eluting the digestion product from the nanoporous film using a biological buffer; and (d) detecting the presence of the at least one proteolytic fragment of an ESAT-6-, CFP-10-, or IF-10-specific protein or peptide in the eluted sample via a conventional analytical quantitation method such as MS.

### BRIEF DESCRIPTION OF THE DRAWINGS

For promoting an understanding of the principles of the invention, reference will now be made to the embodiments, or examples, illustrated in the drawings and specific language will be used to describe the same. It will, nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates.

The following drawings form part of the present specification and are included to demonstrate certain aspects of the present invention. The application contains at least one drawing that is executed in color. Copies of this patent or patent application publication with color drawing(s) will be provided by the Patent and Trademark Office upon request and payment of the necessary fee. The invention may be better understood by reference to the following description taken in conjunction with the accompanying drawings, in which like reference numerals identify like elements, and in which:

FIG. 1A, FIG. 1B, and FIG. 1C show a schematic of an experimental procedure for one embodiment. The biological fluid was fractionated by nanoporous silica film coated on the substrates. (i) the large-abundance proteins were excluded by 5- to 8-nm sized pores; (ii) after extensive washing, relatively small ESAT-6/CFP-10 was retained in the nanopore; (iii) the enzyme, trypsin, digested the ESAT-6/CFP-10 into small fragments; (iv) the protein fragments were eluted in elution buffer, and then detected by MS (FIG. 1A); fingerprint mass spectrum of CFP-10 fragment after trypsin digestion (FIG. 1B) four major fragments of CFP-10, mass 1142.6276 Da and 1317.6645 Da, 1593.7503 and 2003.9781 ([M+H]⁺) exhibit high signals in MALDI MS); and fingerprint mass spectrum of ESAT-6 fragment after trypsin digestion (FIG. 1C) two major fragments of ESAT-6, mass 1900.9511 Da and 1907.9246 Da ([M+H]⁺) exhibit high signals in MALDI-TOF MS;

FIG. 2A, FIG. 2B, FIG. 2C and FIG. 2D show the mass spectra of ESAT-6 in human serum (ESAT-6=80 μM). Spectra were zoomed to show the 1901 peak in the insets (FIG. 2A). Serum mixed with ESAT-6 was directly digested with trypsin without on-chip fractionation. High-abundance proteins hindered the signal of ESAT-6 (FIG. 2B). The serum was fractionated with NSCs, and then treated with trypsin. The efficiencies of fractionation and digestion process in NSCs with different pore sizes (ESAT-6=80 μM) were determined: FIG. 2C shows the intensity of 1901 fragments of ESAT-6. The highest signal was observed in the NSCs with 6-nm pore diameter; FIG. 2D shows the intensity of the 1901 fragment normalized by the number of pores per surface area;

FIG. 3A, FIG. 3B, FIG. 3C and FIG. 3D show BET and ellipsometry was used to measure Film characteristics and dimensions; FIG. 3A: The porosity and film thickness were measured by ellipsometry. The surface area, pore volume, and pore size were determined by N₂ adsorption/desorption analysis. The L121+25% PPG (thin) was expected to have the same pore morphology as standard L121+25% PPG; FIG. 3B: The proportion of un-fragmented CFP-10 that was retained in the detection well after washing (40 ng of CFP-10 was applied in a 7-mm² sized well, mean±s.d., n=6). L121+25% PPG could isolate up to 36 ng; FIG. 3C: MALDI MS signal intensity of each CFP-10 fragment normalized to its own isotopic fragments. Recombinant CFP-10 was spiked into the culture media, which was then treated by on-chip fractionation and digestion prior to MS analysis (mean±s.d.; n=5); FIG. 3D: Measuring the amount of CFP-10 fragments recovered from sample input. Recombinant CFP-10 (40 ng) was spiked into the culture medium, which was then treated with on-chip fractionation and digestion. The absolute amounts of CFP-10 fragments (1142.63 and 1593.75 [M+H]⁺) were quantified by spiking isotopic fragment into eluted samples;

FIG. 4 shows the depth profiles of CFP-10 enriched on L121+25% PPG, as determined from the N1s spectrum collected using XPS. The line represent the exponential fit of y=y0+A^{(-x/B)}+C^{(-x/D)}. CFP-10 could penetrate 100 nm into the film. The inset shows representative XPS N1S spectra of nanoporous films with and without CFP-10;

FIG. 5 shows the relative intensity of each major CFP-10 fragment to its isotopic fragment is plotted verses the input CFP-10 concentration. The isotopic ¹⁸O-labeled fragments were generated by trypsin digestion in H₂¹⁸O. Isotopic CFP-10 at 42 nM of was added in equal proportion to known digested CFP-10 before spiking on MALDI MS plate. In this condition, the 1142.63 and 1593.75 fragments show good linear relation with their respective isotopic fragments below 400 nM;

FIG. 6A, FIG. 6B, and FIG. 6C show different amounts of recombinant ESAT-6 in urine (mean±s.d., n=6), and recombinant CFP-10 in MTB culture media (mean±s.d.; n=5). FIG. 6A: The red line represents the fit of y=ax/(1+ax). The semi-log plot was presented in the inset. The 1901 fragment was detected when the concentration of ESAT-6 was above 60 nM. The detection threshold for CFP-10 fragments by MALDI-TOF MS analysis: The signals of each fragment were normalized by its own isotope as an internal standard; FIG. 6B: Un-precipitated culture medium for each CFP-10 dilution was processed through on-chip fractionation and digestion. The sensitivity plot maintained good linear regression above 13.4 nM in log-log scale; FIG. 6C: the samples were precipitated 10× by ammonium sulfate prior to on-chip processing. MS analysis showed the detection limit had been lowered to 1.3 nM because of sample concentration;

FIG. 7 shows the mass spectra of MTB-specific CFP-10 fragments. None of these fragments was observed in the culture of non-TB specie of mycobacteria (Mycobacterium avium);

FIG. 8 shows the intensity of 1901 fragment obtained by MALDI MS at different ESAT-6 concentration in human serum (mean±s.d., n=6). The red line represents the fit of y=ax/(1+ax). The semi-log plot is presented in the inset. The 1901 fragment was detectable when the ESAT-6 concentration was above 60 nM;

FIG. 9 shows the fingerprint mass-spectrum of full-length recombinant ESAT-6 and full-length recombinant CFP-10 collected in linear mode of MALDI-TOF MS at 5 μM concentration. The molecular weights of recombinant ESAT-6 and recombinant CFP-10 were 13 kDa and 11 kDa, respectively;

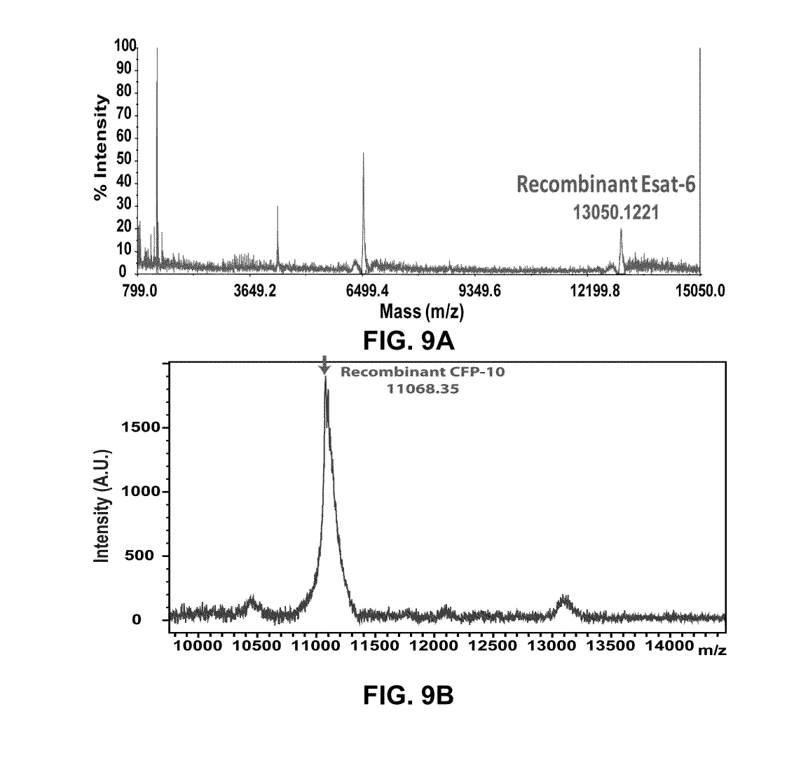

FIG. 10 shows the MALDI MS of human serum treated with on-chip fractionation and trypsin digestion. No peak was selected in the range from 1895 to 1910 (Signal-to-noise ratio threshold=3, noise-window-width=250 in DataExplorer® software). The fragment from human serum did not overlap with ESAT-6 fragments at 1900.9511 Da ([M+H]⁺)

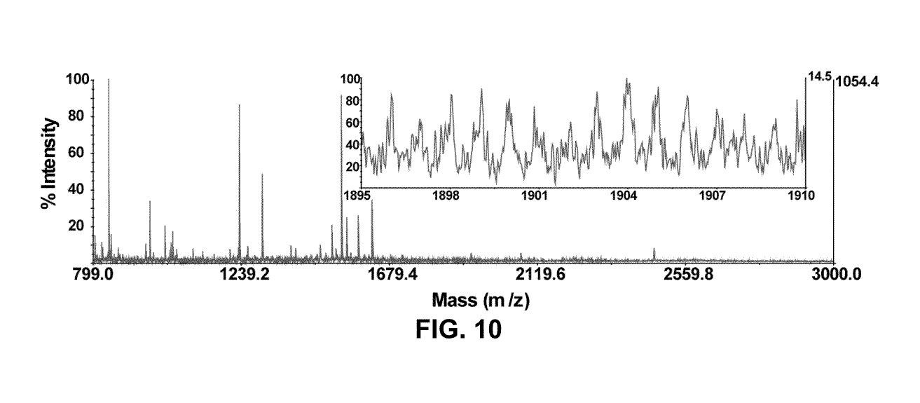

FIG. 11 illustrates the XPS depth profiles of ESAT-6 enriched in nanopores of 6- and 8-nm. The amount of ESAT-6 was determined from N1s spectra collected by XPS. The lines represented the exponential fit of y=y0+A^{(-x/B)}. ESAT-6 penetrated deeper in the 8-nm nanopore because of the slower decay of depth profile (B=29 and 48 nm for 6- and 8-nm sized nanopores, respectively). The total amount of ESAT-6 trapped in the 6-nm pore was higher than 8-nm NSC because there were more nanopores per surface area in 6-nm NSC. The inset represents the depth profiles normalized to the number of pore per surface area. After normalization, more ESAT-6 antigens were trapped in 8-nm NSC;

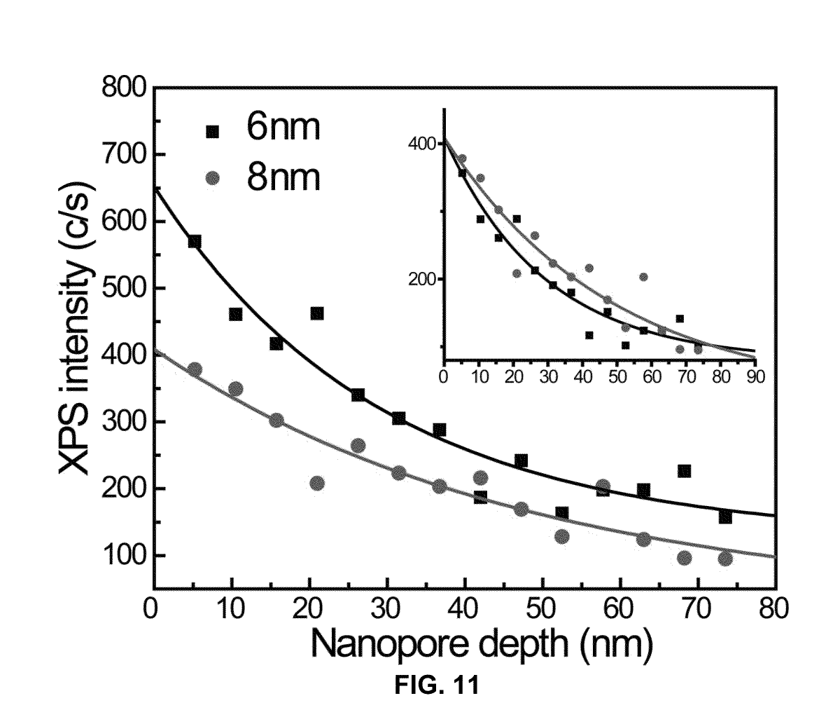

FIG. 12 illustrates the improvement of ESAT-6 signal with a pre-concentration procedure. 1 mL of 40-nM ESAT-6 in urine was concentrated by ammonium sulfate precipitation procedure. The precipitated proteins were dissolved in a final volume of 20 μL buffer. Compared to a non-concentrated sample (250 nM ESAT-6), the precipitation procedure significantly improved the signal of ESAT-6;

FIG. 13 shows the indirect ELISA standard curve of ESAT-6 in 1×PBS, urine, and 5% diluted human serum. Indirect ELISA could achieve a higher sensitivity in the samples with low background proteins. In 1×PBS solution, an ESAT-6 signal was observed at 2 nM concentration. In 100% human serum, signals below micromolar concentration could not be observed;

FIG. 14 shows the fingerprinting spectra of ESAT-6 and CFP-10 fragments. Two major fragments of ESAT-6 were observed in MALDI-TOF MS. Compared to ESAT-6, five fragments of CFP-10 were observed;

FIG. 15 shows the spectrum of human serum containing ESAT-6 and CFP-10 antigens after treated with on-chip fractionation. Both CFP-10 and ESAT-6 fragments could be observed simultaneously. CFP-10 showed higher intensity than ESAT-6 in MALDI-TOF MS;

FIG. 16 shows the dual-layer NSC photo collected from scanning electron microscope. The top layer of the nanoporous film was made by L121+25% PPG of ˜90 nm thickness, and the bottom layer was made by L121 with ˜700-nm thickness; and

FIG. 17A and FIG. 17B show the spectra of human serum containing ESAT-6 and CFP-10 antigens as low as 100 nM after treated with ultracentrifugation coupling with on-chip fractionation. Two CFP-10 fragments with m/z 1593.756 and 2003.989 showed clear signals in MALDI-TOF MS after treated with single-layer NSC (L121+25% PPG) (FIG. 17A) or dual-layer NSC (L121+25% PPG/L121) (FIG. 17B).

### BRIEF DESCRIPTION OF THE AMINO ACID SEQUENCES

SEQ ID NO:1 is an exemplary ESAT-6-specific peptide sequence for use in accordance with one aspect of the present invention.

SEQ ID NO:2 is an exemplary ESAT-6-specific peptide sequence for use in accordance with one aspect of the present invention.

SEQ ID NO:3 is an exemplary CFP-10-specific peptide sequence for use in accordance with one aspect of the present invention.

SEQ ID NO:4 is an exemplary CFP-10-specific peptide sequence for use in accordance with one aspect of the present invention.

SEQ ID NO:5 is an exemplary CFP-10-specific peptide sequence for use in accordance with one aspect of the present invention.

SEQ ID NO:6 is an exemplary CFP-10-specific peptide sequence for use in accordance with one aspect of the present invention.

SEQ ID NO:7 is an exemplary CFP-10-specific peptide sequence for use in accordance with one aspect of the present invention.

### DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would be a routine undertaking for those of ordinary skill in the art having the benefit of this disclosure.

Detection of M. Tuberculosis in Biological Samples

Various embodiments described herein relate to the detection of one or more M. tuberculosis-specific biomolecules in a sample. The sample can comprise, or be derived from, at least one biological fluid selected from the group consisting of blood serum, blood plasma, blood, urine, seminal fluid, seminal plasma, pleural fluid, ascites, nipple aspirate, feces, and saliva (see, for example, U.S. Patent Appl. Publ. No. 2011/0065201, specifically incorporated herein in its entirety by express reference thereto). In particular, the sample may contain, or be derived from, at least one bodily fluid, including, without limitation, sputum, pleural effusion, cerebrospinal fluids, urine, serum and plasma. Preferably the bodily fluid will be obtained from a vertebrate mammal, and in particular, a human having, suspected of having, and/or at risk for developing a bacterial infection, such as that caused by one or more strains or species of Mycobacteria.

Many M. tuberculosis-specific biomolecules are suitable for detection by the methods described herein, including, for example, proteins, peptides, polynucleotides, and polysaccharides. In exemplary embodiments, a sample will preferably contain at least one ESAT-6-specific protein or peptide or at least one CFP-10-specific protein or peptide (or one or more proteolytic products thereof), each of which has been shown to be a specific biomarker for TB (see e.g., Collins et al., 2005; and Flores et al., 2011; each of which is specifically incorporated herein in its entirety by express reference thereto).

Nanoporous Films

Various nanoporous films and fabrication methods therefor suitable for use in the practice of the invention have been recently described in, for example, Bouamrani et al., 2010; Hu et al., 2010; Hu et al., 2011; and U.S. Pat. Appl. Publ. No. 2011/0065207; each of which is incorporated herein in its entirety by express reference thereto).

The nanoporous film can comprise, for example, a plurality of pores, including mesopores having an average diameter of about 2 to about 50 nm. In some embodiments, the nanoporous film comprises a plurality of mesopores having an average diameter of about 2 to about 20 nm, preferably about 3 to about 10 nm, or more preferably about 4 to about 8 nm. In some embodiments, the nanoporous film may include pores of substantially the same diameter, while in other embodiments, the nanoporous film may include pores of two or more distinctly different average diameters.

The porosity of the nanoporous films of the present invention can be, for example, at least about 40 to about 90%, alternatively, at least about 50 to about 80%, or more preferably still, at least about 60 to about 70%. The pore morphology can be pre-determined, for example, to include cubic, hexagonal, honeycomb-like, tubular, circular, or oblong pores, and/or one or more combinations thereof. In some embodiments, the nanoporous films of the invention may include at least two distinct domains, each including pores of substantially differing sizes, connectivities, and/or morphologies.

In particular embodiments, the nanoporous films are net positively-charged, to facilitate attraction to negatively-charged biomolecules. In other embodiments, the nanoporous films may be net negatively-charged, and therefore useful in attracting positively-charged biomolecules. In alternative embodiments, the nanoporous film may be fabricated such that it is substantially electrically net neutral.

Optionally, the nanoporous films of the invention may be fabricated to contain, for example, one or more nanoporous oxide materials (including, for example, nanoporous silica, nanoporous titanium oxide, nanoporous alumina, nanoporous iron oxide, nanoporous silicon, nanoporous carbon, or any combination thereof). The nanoporous films of the invention may also be functionalized with one or more organic functional groups, one or more metal ions, or combinations thereof using conventional methodologies (see, e.g., U.S. Pat. Appl. Publ. No. 2011/0065207, which is specifically incorporated in its entirety by express reference thereto).

The nanoporous film may, for example, be composed of a single-layer nanoporous film, a dual-layer nanoporous film, or even a multi-layer nanoporous film. Dual-layer nanoporous films may include, for example, a first or bottom layer having a first average pore diameter, and a second or top layer having a second average pore diameter that is distinct (e.g., larger than) from the first average pore diameter of the first layer. Similarly, a multi-layer nanoporous film may include, for example, a third distinct layer, having a third average pore diameter that is distinct from the average pore diameters of the first and/or second layers. Such dual- and multi-layer nanoporous films may be fabricated, for example, by serially or sequentially coating two or more different silicate sol solutions on a single substrate to build up the multi-layer film in a layer-by-layer fashion.

In some embodiments, it will be preferable to prepare a dual-layer nanoporous film that includes a top layer having a larger average pore size, and a bottom layer having a smaller average pore size, to enhance the capillary force of the top layer nanoporous film having the larger pore sizes. One or more full-length antigens may be trapped, for example, in the top layer of such a dual-layer nanoporous film. After washing, a digestion buffer comprising one or more proteolytic enzymes can be applied to digest the full-length antigens into smaller fragments. At least some of the smaller antigenic digestion fragments can then trapped, for example, in the bottom layer of the nanoporous film, having flowed through to the bottom layer from the top layer.

Optionally, a second wash can be applied to remove the enzyme and certain salts of the digestion buffer. The antigen fragments will remain in the bottom layer, and can then be removed using a suitable elution buffer. In some embodiments, sensitivity of the methods described herein can be improved by using dual-rather than single-layer nanoporous films.

The nanoporous film can be fabricated by, for example, a surfactant-templated sol-gel process. The nanoporous film can be fabricated, for example, on a substrate by one or more deposition methods known to those of ordinary skill in the art. The substrate can be, for example, a silicon wafer, glass wafer, or a metal layer. The nanoporous film can be deposited onto the surface by one or more methods known to those of ordinary skill in the art, including, without limitation, by spin-coating, by dip-coating, or a combination thereof.

In some embodiments, nanoporous film is fabricated from a coating solution comprising at least one silicate sol, at least one tri-block copolymer, and at least one swelling agent, and at least one solvent. The coating solution can be deposited on a silicon wafer by a conventional method, including, for example, spin coating, dip-coating, or the like. The preferential evaporation of the solvent after spin coating or dip-coating drives silica/copolymer self-assembly into a uniform thin film nanophase by increasing the concentration of polymer to exceed the critical micelle concentration. After removing the polymer template by calcination, nanoporous films with narrow nanoscale pore size distribution and high ratio of surface area to pore volume are formed. Optionally, oxygen plasma treatment can be performed to modify the surface of the nanoporous film.

Further, to facilitate the application of samples, at least one gasket can be attached on top of the nanoporous film. The use of gaskets in nanoporous film fabrication is known to those of ordinary skill in the art, and such gaskets can be made of any suitable material, such as, for example, silicone, metal, rubber, fiberglass, polymer, and the like. In some embodiments, a silicone gasket may be used. Such a gasket may contain, for example, a plurality of culture wells. In preferred embodiments, each culture well is fabricated to provide a diameter of about 3-mm, and a height of about 1-mm, although other culture well dimensions are contemplated to fall within the scope of the present disclosure.

Enriching M. Tuberculosis-Specific Biomolecules

In many embodiments described herein, M. tuberculosis-specific biomolecules may be enriched or concentrated using a nanoporous film prior to sample assay and biomarker detection.

In some embodiments, a sample containing one or more M. tuberculosis-specific biomolecules is directly applied onto the nanoporous film. The sample can be applied, for example, into a plurality of culture wells formed by a gasket. In other embodiments, a sample containing one or more M. tuberculosis-specific biomolecules may be indirectly applied onto the nanoporous film through microfluidic channels patterned onto the nanoporous film (see, e.g., Hu et al., 2011, which is specifically incorporated herein in its entirety by express reference thereto).

Upon contacting the nanoporous film, the M. tuberculosis-specific biomolecules, due to their size, will be able to enter and reside within the pores of the nanoporous film. The M. tuberculosis-specific biomolecules can be absorbed, for example, on the walls of one or more such pores. The M. tuberculosis-specific biomolecules can absorbed onto the nanoporous film, for example, by van der Waals forces. When the M. tuberculosis-specific biomolecules and the pores are of opposite charge, electrostatic interaction between the target and the film itself is facilitated. The use of oppositely-charged films may increase the overall yield or rate of adsorption, but is not required.

In one embodiment, the nanoporous film may be adapted and configured such that the abundant serum protein, albumin, is substantially excluded from entering the pores. After the sample suspected of containing M. tuberculosis-specific biomolecules is applied to the nanoporous film, the film may be washed one or more times to remove large molecules such as albumin that were not collected and enriched by the nanoporous film. In one embodiment, water may be used for the washing step.

In some embodiments, the sample suspected of containing one or more M. tuberculosis-specific biomolecules may be “pre-concentrated” before application onto the nanoporous film. M. tuberculosis-specific biomolecules, if present in the sample, can be pre-concentrated by one or more conventional methods, including for example, by precipitating the proteinaceous fraction of the sample. Standard protein precipitation methods are known to those of ordinary in the art, and may include, for example, the use of ammonium sulfate. The resulting protein precipitate can then be dissolved in a suitable solvent before being applied onto the nanoporous film.

Proteolysis of M. tuberculosis-Specific Biomolecules

In many embodiments described herein, a sample suspected of containing M. tuberculosis-specific biomolecules may be subjected to proteolysis prior to the step of detecting the molecules in the sample. For example, the sample may be digested using one or more proteolytic enzymes (e.g., a protease or a peptidase), to enzymatically-cleave one or more proteins present in the sample. In embodiments wherein the biomolecule of interest is an M. tuberculosis-specific protein or peptide, the sample may be pre-treated with one or more proteases.

Various protease or peptidase are known to those of ordinary skill in the art, including, for example, serine proteases, cysteine proteases, aspartate proteases, threonine proteases, glutamic acid proteases, metalloproteases, and such like (see e.g., Rawlings, et al., 2010). In particular embodiments, the protease is preferably trypsin or an analog or active fragment thereof.

In some embodiments, the M. tuberculosis-specific biomolecule can be digested directly on or within the nanoporous film itself (i.e., “on-chip digestion” or on-site proteolysis). The proteolytic products so produced can then be extracted from the nanoporous film for further characterization or analysis. Extraction of the digestion products may be achieved by, for example, using one or more suitable elution buffers. In other embodiments, the biomolecules of interest, or the proteolytic byproducts thereof may be extracted from the nanoporous film before being digested at a different site.

The digestion product can comprise, for example, at least one, at least two, or least three different identifiable fragments of a M. tuberculosis-specific biomolecule. In some embodiments where the M. tuberculosis-specific biomolecule is a protein or peptide, the digestion product can comprise, for example, at least one, at least two, or least three different identifiable peptides resulting from proteolytic cleavage of the molecule.

In some embodiments where the M. tuberculosis-specific biomolecule is an ESAT-6 protein or peptide, the digestion product can comprise, for example, at least one peptide having a mass fingerprint at about 1895-1910 Da. The digestion product(s) can include, for example, at least one ESAT-6 fragment comprising, consisting essentially of, or alternatively, consisting of, the sequence of WDATATELNNALQNLAR (SEQ ID NO:1), at least one ESAT-6 fragment comprising, consisting essentially of, or alternatively, consisting of, the sequence of LAAAWGGSGSEAYQGVQQK (SEQ ID NO:2), or a combination of both fragments.

In some embodiments, wherein the M. tuberculosis-specific biomolecule is a CFP-10 protein or peptide, the digestion product can include, for example, at least one CFP-10 fragment resulting from proteolysis. In such embodiments, the digestion product(s) can include, for example, at least one CFP-10-specific peptide fragment that comprises, consists essentially of, or alternatively, consists of, the amino acid sequence of any one of: TQIDQVESTAGSLQGQWR (SEQ ID NO:3), ADEEQQQALSSQMGF (SEQ ID NO:4), TDAATLAQEAGNFER (SEQ ID NO:5), GAAGTAAQAAVVR (SEQ ID NO:6) and QAGVQYSR (SEQ ID NO:7). In certain embodiments, the digestion products can include, for example, at least two CFP-10-specific peptide fragments, each of which can comprise, consist essentially of, or alternatively, consist of, an amino acid sequence as set forth in any one of SEQ ID NO:3, SEQ ID NO:4, SEQ ID NO:5, SEQ ID NO:6 and SEQ ID NO:7.

Likewise, in other embodiments, the digestion products can include, for example, at least three, at least four, or at least CFP-10-specific peptide fragments, each of which comprising, consisting essentially of, or alternatively, consisting of, an amino acid sequence as set forth in any one of SEQ ID NO:3, SEQ ID NO:4, SEQ ID NO:5, SEQ ID NO:6 and SEQ ID NO:7.

Detecting M. tuberculosis-Specific Biomolecules

In many embodiments described herein, the presence of a M. tuberculosis-specific biomolecule can be detected by any one or more techniques known to those of ordinary skill in the art, including, without limitation, MS, gel electrophoresis, chromatography, one or more bioassays, one or more immunological assays, or a combination of two or more such techniques. In the practice of the invention, MS has been preferably used to detect the presence of M. tuberculosis-specific proteins and peptides from a sample of interest. Exemplary MS methods include, without limitation, matrix-assisted laser desorption/ionization time-of-flight (MALDI-TOF) MS, liquid chromatography-MS (LC-MS), electrospray ionization (ESI)-MS (ESI-MS), tandem-MS, and/or surface-enhanced laser desorption/ionization (SELDI)-MS (SELDI-MS).

In one embodiment, MALDI-TOF MS may be employed to readily detect the presence of one or more M. tuberculosis-specific biomolecules. In another embodiment, SELDI is used to detect the presence of the M. tuberculosis-specific biomolecule. In a further embodiment, LC-MS is used to detect the presence of a M. tuberculosis-specific biomolecule.

The presence of a M. tuberculosis-specific biomolecule can be detected by, for example, finding at least one, at least two, or at least three or MS “fingerprints” unique to the particular biomolecule of interest. The presence of one or more M. tuberculosis-specific biomolecules in a sample can be detected by, for example, finding at least one, at least two, or at least three or more mass fingerprints of one or more enzymatic digestion products of the particular biomolecule of interest.

In some embodiments, wherein the M. tuberculosis-specific biomolecule is an ESAT-6-specific protein or peptide, the presence of ESAT-6 can be detected by, for example, finding at least one mass fingerprint of an ESAT-6 protein or one or more of its proteolytic fragments. The presence of an ESAT-6-specific molecule can be detected by, for example, identifying a mass fingerprint at about 1895-1910 Da, or more precisely, a mass fingerprint at about 1900.9511 Da ([M+H]⁺) Similarly, the presence of an ESAT-6-specific molecule can be detected by, for example, identifying a mass fingerprint at about 1907.9246 Da ([M+H]⁺)

Alternatively, where the M. tuberculosis-specific biomolecule is a CFP-10-specific protein or peptide, the presence of the CFP-10 biomarker can be detected by, for example, identifying at least one mass fingerprint of a CFP-10 protein or one or more of its proteolytic fragments. The presence of CFP-10 can be detected by, for example, identifying a mass fingerprint at about 2003.9781 Da ([M+H]⁺) at about 1668.7170 Da ([M+H]⁺) or at about 1593.7503 Da ([M+H]⁺). The presence of a CFP-10-specific protein or peptide can also be confirmed by identifying, for example, a mass fingerprint at about 1142.6276 Da ([M+H]⁺) or at about 908.4584 Da ([M+H]⁺)

In one embodiment, at least one M. tuberculosis-specific biomolecule is detected to identify active TB, such as ESAT-6 or CFP-10. In other embodiments, at least two M. tuberculosis-specific biomolecules may be detected within a sample to identify the presence of TB organisms, such as ESAT-6 and CFP-10. In further embodiments, three or more M. tuberculosis-specific biomolecules may be detected to confirm the presence of TB in a sample.

Exemplary Methods

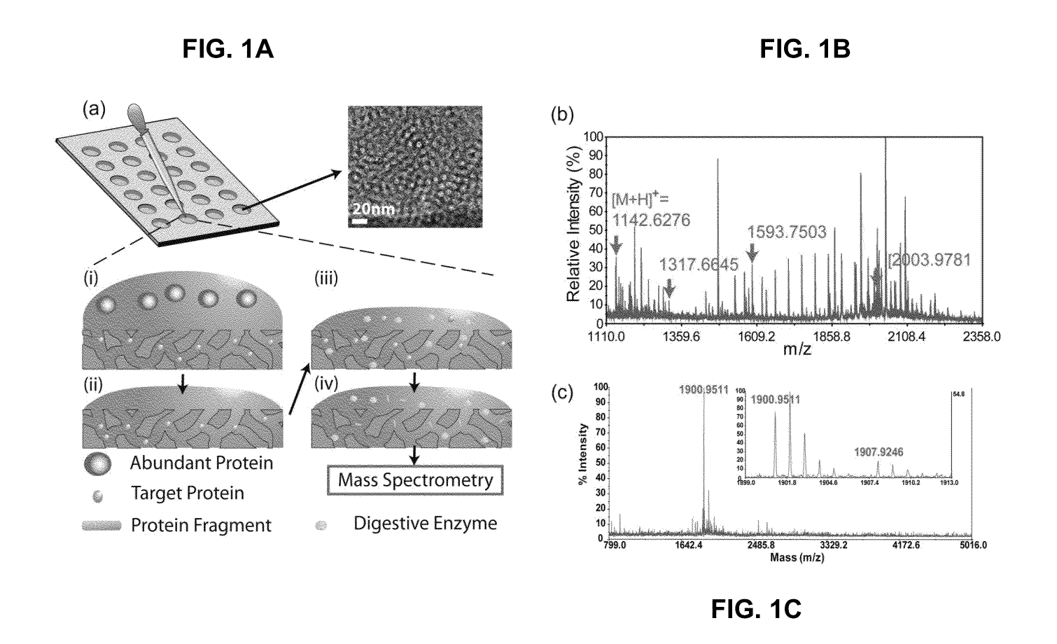

FIG. 1A illustrates an exemplary detection procedure for ESAT-6 and/or CFP-10. The biological samples can be applied to a gasket culture well attached on top of a nanoporous silica film. The nanoporous silica films can be fixed on a flat substrate. The fractionation process can be completed after serial washes. The relatively small size of ESAT-6 (molecular weight: 10 kDa) allows it to be captured by the silica nanopores that have an average diameter in the range of about 5, about 6, about 7 or about 8 or so nm.

To identify ESAT-6 and/or CFP-10 from biological fluids with MALDI-TOF MS, mass fingerprinting may be performed. Because smaller proteins or peptide species provide higher signals and resolution in MS, a proteolytic enzyme, such as trypsin, may be used to pre-treat the sample, and to cleave full-length ESAT-6 and/or CFP-10 proteins into smaller peptide sub-fragments, which can then be detected by suitable methods.

One advantage of the instant method is that unlike conventional digestion processes, which are usually conducted in bulk solution, the proteolytic enzyme(s) can be applied directly onto the nanoporous silica film, where they can interact with the sample inside the nanopores. This on-chip nanobiocatalysis process provides many advantages over solution-based proteolysis, including higher efficiency and better stability. In addition, the on-chip digestion protocol can eliminate several additional steps, including protein extraction from the silica nanopore and buffer exchange for enzymatic digestion, and thus further simplify the overall diagnostic assay.

Additional Applications

While the present invention has been optimized to detect biomolecules that are specific for MT and the detection of TB-causing organisms, the methods and apparatus described herein can also be used to detect other biomolecules of interest.

In certain applications, the target protein is specific to one or more pathogens associated with a particular infectious disease. In a manner analogous to that demonstrated herein for ESAT-6 and CFP-10 proteins, the particular pathogen-specific protein of interest may be digested with one or more proteases to produce one or more peptide fragments, at least one of which comprises a unique mass spectral fingerprint, and those fingerprints can be detected via MS, such as MALDI-TOF MS.

The methods and kits described herein possess broad applicability in the molecular arts, since many infectious diseases have been linked to specific microorganisms, many of which have known type- or species-specific biomarkers, which may be detected in a manner analogous to that demonstrated herein for TB-specific biomarkers. Exemplary pathogens suitable for detection using the disclosed methods include, but are not limited to, bacterial pathogens, viral pathogens, fungal pathogens, unicellular eukaryotic pathogens such as protozoans, spirochetes, prions, or other pathogenic microbiological organisms.

Specific examples include, without limitation, the detection of viral pathogens such as HSV, HIV, West Nile Virus, hantavirus, Hepatitis A, Hepatitis B, Norovirus, poliovirus, Rotavirus, etc., the detection of bacterial pathogens such as the causal agents of pneumonia, Legionnaire's disease, food poisoning, food infection, food intoxication, diphtheria, Lyme disease, and/or the detection of protozoal pathogens, including, without limitation, those of the genus Plasmodium.

Diagnostic Kits

Kits including one or more of the disclosed pathogen-specific biomarkers or pharmaceutical formulations including such; and instructions for using the kit in a diagnostic, therapeutic, prophylactic, and/or other clinical embodiment(s) also represent preferred aspects of the present disclosure. Such kits may include one or more of the disclosed pathogen-specific biomarkers, either alone, or in combination with one or more additional diagnostic compounds, pharmaceuticals, and such like. The kits according to the invention may be packaged for commercial distribution, and may further optionally include one or more delivery, storage, or assay components.

The container(s) for such kits may typically include at least one vial, test tube, flask, bottle, syringe, or other container, into which the pathogen-specific biomarker composition(s) may be placed. Alternatively, a plurality of distinct biomarker composition(s) and/or distinct proteolytic enzymes may be prepared in a single formulation, and may be packaged in a single container, vial, flask, syringe, bottle, test tube, ampoule, or other suitable container. The kit may also include a larger container, such as a case, that includes the containers noted above, along with other equipment, instructions, and the like.

For example, a kit can be provided that includes at least two of the following components:

(i) a nanoporous film disposed on a solid substrate adapted for accepting a human body fluid sample and enriching at least one target protein therefrom, wherein the nanoporous film comprises a plurality of pores in which the enriched target protein resides,

(ii) a digestion buffer comprising at least one protease adapted for digesting the target protein to produce at least one fragment of the target protein having a mass fingerprint detectable in MS,

(iii) an elution buffer adapted for extracting the at least one fragment of the target protein from the nanoporous film,

(iv) a washing buffer adapted for washing the nanoporous film before the digestion buffer is added onto the nanoporous film, and

(v) instructions for using the kit.

The nanoporous film can be, for example, a silica film comprising a plurality of pores having an average diameter of about 3 to about 10 nm, and more preferably, a silica film comprising a plurality of pores having an average diameter of about 6 to about 8 nm, wherein the digestion buffer comprises a first proteolytic enzyme, such as trypsin, and wherein the kit comprises at least one gasket attached onto the nanoporous film to form a plurality of wells.

One embodiment, for example, provides a kit, comprising (i) a nanoporous film disposed on a solid substrate adapted for accepting a sample of a human bodily fluid, and enriching at least one target biomarker protein or peptide therefrom, wherein the nanoporous film comprises a plurality of pores in which the enriched target protein resides, and (ii) a digestion buffer comprising at least one protease adapted for digesting the target protein to produce at least one fragment of the target protein having a mass fingerprint detectable in MS. In one embodiment, the kit can further comprise an elution buffer adapted for extracting the at least one fragment of the target protein from the nanoporous film. In one embodiment, the kit can further comprise a washing buffer adapted for washing the nanoporous film before the digestion buffer is added onto the nanoporous film.

The kit can be part of a larger system. For example, the system can also include an instrument such as, for example, a MS device for detecting said mass fingerprint. Sample preparation items can also be included in the various systems and kits.

Exemplary Definitions

Unless defined otherwise, all technical and scientific terms used herein have the same meaning as commonly understood by one of ordinary skill in the art to which this invention belongs. Although any methods and compositions similar or equivalent to those described herein can be used in the practice or testing of the present invention, the preferred methods, and compositions are described herein. For purposes of the present invention, the following terms are defined below:

In accordance with long-standing patent law convention, the words “a” and “an,” when used in this application (including in the appended claims), denotes “one or more.”

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

As used herein, an “antigenic polypeptide” or an “immunogenic polypeptide” is a polypeptide which, when introduced into a vertebrate, reacts with the vertebrate's immune system molecules, i.e., is antigenic, and/or induces an immune response in the vertebrate, i.e., is immunogenic.

As used herein, the term “buffer” includes one or more compositions, or aqueous solutions thereof, that resist fluctuation in the pH when an acid or an alkali is added to the solution or composition that includes the buffer. This resistance to pH change is due to the buffering properties of such solutions, and may be a function of one or more specific compounds included in the composition. Thus, solutions or other compositions exhibiting buffering activity are referred to as buffers or buffer solutions. Buffers generally do not have an unlimited ability to maintain the pH of a solution or composition; rather, they are typically able to maintain the pH within certain ranges, for example from a pH of about 5 to 7.

As used herein, the term “carrier” is intended to include any solvent(s), dispersion medium, coating(s), diluent(s), buffer(s), isotonic agent(s), solution(s), suspension(s), colloid(s), inert(s), or such like, or a combination thereof that is pharmaceutically acceptable for administration to the relevant animal or acceptable for a therapeutic or diagnostic purpose, as applicable.

As used herein, the term “DNA segment” refers to a DNA molecule that has been isolated free of total genomic DNA of a particular species. Therefore, a DNA segment obtained from a biological sample using one of the compositions disclosed herein refers to one or more DNA segments that have been isolated away from, or purified free from, total genomic DNA of the particular species from which they are obtained. Included within the term “DNA segment,” are DNA segments and smaller fragments of such segments, as well as recombinant vectors, including, for example, plasmids, cosmids, phage, viruses, and the like.

As used herein, “an effective amount” would be understood by those of ordinary skill in the art to provide a therapeutic, prophylactic, or otherwise beneficial effect against the organism, its infection, or the symptoms of the organism or its infection, or any combination thereof.

The term “e.g.,” as used herein, is used merely by way of example, without limitation intended, and should not be construed as referring only those items explicitly enumerated in the specification.

As used herein, the terms “engineered” and “recombinant” cells are intended to refer to a cell into which an exogenous polynucleotide segment (such as DNA segment that leads to the transcription of a biologically active molecule) has been introduced. Therefore, engineered cells are distinguishable from naturally occurring cells, which do not contain a recombinantly introduced exogenous DNA segment. Engineered cells are, therefore, cells that comprise at least one or more heterologous polynucleotide segments introduced through the hand of man.

As used herein, the term “epitope” refers to that portion of a given immunogenic substance that is the target of, i.e., is bound by, an antibody or cell-surface receptor of a host immune system that has mounted an immune response to the given immunogenic substance as determined by any method known in the art. Further, an epitope may be defined as a portion of an immunogenic substance that elicits an antibody response or induces a T-cell response in an animal, as determined by any method available in the art (see, for example, Geysen et al., 1984). An epitope can be a portion of any immunogenic substance, such as a protein, polynucleotide, polysaccharide, an organic or inorganic chemical, or any combination thereof. The term “epitope” may also be used interchangeably with “antigenic determinant” or “antigenic determinant site.”

As used herein, “heterologous” is defined in relation to a predetermined referenced DNA or amino acid sequence. For example, with respect to a structural gene sequence, a heterologous promoter is defined as a promoter that does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

As used herein, the term “homology” refers to a degree of complementarity between two polynucleotide or polypeptide sequences. The word “identity” may substitute for the word “homology” when a first nucleic acid or amino acid sequence has the exact same primary sequence as a second nucleic acid or amino acid sequence. Sequence homology and sequence identity can be determined by analyzing two or more sequences using algorithms and computer programs known in the art. Such methods may be used to assess whether a given sequence is identical or homologous to another selected sequence.

As used herein, “homologous” means, when referring to polypeptides or polynucleotides, sequences that have the same essential structure, despite arising from different origins. Typically, homologous proteins are derived from closely related genetic sequences, or genes. By contrast, an “analogous” polypeptide is one that shares the same function with a polypeptide from a different species or organism, but has a significantly different form to accomplish that function. Analogous proteins typically derive from genes that are not closely related.

The terms “identical” or percent “identity,” in the context of two or more peptide sequences, refer to two or more sequences or subsequences that are the same or have a specified percentage of amino acid residues that are the same, when compared and aligned for maximum correspondence over a comparison window, as measured using a sequence comparison algorithm or by manual alignment and visual inspection.

The phrases “isolated” or “biologically pure” refer to material that is substantially, or essentially, free from components that normally accompany the material as it is found in its native state. Thus, an isolated peptide in accordance with the invention preferably does not contain materials normally associated with that peptide in its in situ environment.

As used herein, the term “kit” may be used to describe variations of the portable, self-contained enclosure that includes at least one set of reagents, components, or pharmaceutically-formulated compositions to conduct one or more of the diagnostic methods of the invention.

“Link” or “join” refers to any method known in the art for functionally connecting peptides, including, without limitation, recombinant fusion, covalent bonding, disulfide bonding, ionic bonding, hydrogen bonding, electrostatic bonding, and such like.

As used herein, “mammal” refers to the class of warm-blooded vertebrate animals that have, in the female, milk-secreting organs for feeding the young. Mammals include without limitation humans, apes, many four-legged animals, whales, dolphins, and bats. A human is a preferred mammal for purposes of the invention.

The term “pathogen” is defined herein as any sort of infectious agent, including e.g., viruses, prions, protozoans, parasites, as well as microbes such as bacteria, yeast, molds, fungi, and the like.

The term “naturally occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally occurring animals.

As used herein, the term “nucleic acid” includes one or more types of: polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. Exemplary nucleic acids include, without limitation, gDNA; hnRNA; mRNA; rRNA, tRNA, micro RNA (miRNA), small interfering RNA (siRNA), small nucleolar RNA (snORNA), small nuclear RNA (snRNA), and small temporal RNA (stRNA), and the like, and any combination thereof.

As used herein, the term “patient” (also interchangeably referred to as “host” or “subject”) refers to any host that can receive one or more of the pharmaceutical compositions disclosed herein. Preferably, the subject is a vertebrate animal, which is intended to denote any animal species (and preferably, a mammalian species such as a human being). In certain embodiments, a “patient” refers to any animal host including without limitation any mammalian host. Preferably, the term refers to any mammalian host, the latter including but not limited to, human and non-human primates, bovines, canines, caprines, cavines, corvines, epines, equines, felines, hircines, lapines, leporines, lupines, murines, ovines, porcines, ranines, racines, vulpines, and the like, including livestock, zoological specimens, exotics, as well as companion animals, pets, and any animal under the care of a veterinary practitioner. A patient can be of any age at which the patient is able to respond to inoculation with the present vaccine by generating an immune response. In certain embodiments, the mammalian patient is preferably human.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures. For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues described herein are preferred to be in the “L” isomeric form. However, residues in the “D” isomeric form may be substituted for any L-amino acid residue provided the desired properties of the polypeptide are retained.

“Protein” is used herein interchangeably with “peptide” and “polypeptide,” and includes both peptides and polypeptides produced synthetically, recombinantly, or in vitro and peptides and polypeptides expressed in vivo after nucleic acid sequences are administered into a host animal or human subject. The term “polypeptide” is preferably intended to refer to all amino acid chain lengths, including those of short peptides of from about 2 to about 20 amino acid residues in length, oligopeptides of from about 10 to about 100 amino acid residues in length, and polypeptides including about 100 amino acid residues or more in length. The term “sequence,” when referring to amino acids, relates to all or a portion of the linear N-terminal to C-terminal order of amino acids within a given amino acid chain, e.g., polypeptide or protein; “subsequence” means any consecutive stretch of amino acids within a sequence, e.g., at least 3 consecutive amino acids within a given protein or polypeptide sequence. With reference to nucleotide and polynucleotide chains, “sequence” and “subsequence” have similar meanings relating to the 5′ to 3′ order of nucleotides.

“Purified,” as used herein, means separated from many other compounds or entities. A compound or entity may be partially purified, substantially purified, or pure. A compound or entity is considered pure when it is removed from substantially all other compounds or entities, i.e., is preferably at least about 90%, more preferably at least about 91%, 92%, 93%, 94%, 95%, 96%, 97%, 98%, 99%, or greater than 99% pure. A partially or substantially purified compound or entity may be removed from at least 50%, at least 60%, at least 70%, or at least 80% of the material with which it is naturally found, e.g., cellular material such as cellular proteins and/or nucleic acids.

The term “sequence,” when referring to amino acids, relates to all or a portion of the linear N-terminal to C-terminal order of amino acids within a given amino acid chain, e.g., polypeptide or protein; “subsequence” means any consecutive stretch of amino acids within a sequence, e.g., at least 3 consecutive amino acids within a given protein or polypeptide sequence. With reference to nucleotide chains, “sequence” and “subsequence” have similar meanings relating to the 5′ to 3′ order of nucleotides.

The term “a sequence essentially as set forth in SEQ ID NO:X” means that the sequence substantially corresponds to a portion of SEQ ID NO:X and has relatively few amino acids (or nucleotides in the case of polynucleotide sequences) that are not identical to, or a biologically functional equivalent of, the amino acids (or nucleic acids) of SEQ ID NO:X. The term “biologically functional equivalent” is well understood in the art, and is further defined in detail herein. Accordingly, sequences that have about 85% to about 90%; or more preferably, about 91% to about 95%; or even more preferably, about 96% to about 99%; of amino acids that are identical or functionally equivalent to one or more of the amino acid sequences provided herein are particularly contemplated to be useful in the practice of the invention and in the detection of pathogen-specific biomarkers from one or more biological samples or specimens.

Suitable standard hybridization conditions for the present invention include, for example, hybridization in 50% formamide, 5×Denhardt's solution, 5×SSC, 25 mM sodium phosphate, 0.1% SDS and 100 μg/mL of denatured salmon sperm DNA at 42° C. for 16 hr followed by 1 hr sequential washes with 0.1×SSC, 0.1% SDS solution at 60° C. to remove the desired amount of background signal. Lower stringency hybridization conditions for the present invention include, for example, hybridization in 35% formamide, 5×Denhardt's solution, 5×SSC, 25 mM sodium phosphate, 0.1% SDS and 100 μg/mL denatured salmon sperm DNA or E. coli DNA at 42° C. for 16 hr followed by sequential washes with 0.8×SSC, 0.1% SDS at 55° C. Those of skill in the art will recognize that conditions can be readily adjusted to obtain the desired level of stringency.

Naturally, the present invention also encompasses nucleic acid segments that are complementary, essentially complementary, and/or substantially complementary to at least one or more of the specific nucleotide sequences specifically set forth herein. Nucleic acid sequences that are “complementary” are those that are capable of base-pairing according to the standard Watson-Crick complementarity rules. As used herein, the term “complementary sequences” means nucleic acid sequences that are substantially complementary, as may be assessed by the same nucleotide comparison set forth above, or as defined as being capable of hybridizing to one or more of the specific nucleic acid segments disclosed herein under relatively stringent conditions such as those described immediately above.

As described above, the probes and primers of the present invention may be of any length. By assigning numeric values to a sequence, for example, the first residue is 1, the second residue is 2, etc., an algorithm defining all probes or primers contained within a given sequence can be proposed:
n to n+y,

where n is an integer from 1 to the last number of the sequence and y is the length of the probe or primer minus one, where n+y does not exceed the last number of the sequence. Thus, for a 25-basepair probe or primer (i.e., a “25 mer”), the collection of probes or primers correspond to bases 1 to 25, bases 2 to 26, bases 3 to 27, bases 4 to 28, and so on over the entire length of the sequence. Similarly, for a 35-basepair probe or primer (i.e., a “35-mer), exemplary primer or probe sequence include, without limitation, sequences corresponding to bases 1 to 35, bases 2 to 36, bases 3 to 37, bases 4 to 38, and so on over the entire length of the sequence. Likewise, for 40-mers, such probes or primers may correspond to the nucleotides from the first basepair to by 40, from the second by of the sequence to by 41, from the third by to by 42, and so forth, while for 50-mers, such probes or primers may correspond to a nucleotide sequence extending from bp 1 to bp 50, from bp 2 to bp 51, from bp 3 to bp 52, from bp 4 to bp 53, and so forth.

The terms “substantially corresponds to,” “substantially homologous,” or “substantial identity,” as used herein, denote a characteristic of a nucleic acid or an amino acid sequence, wherein a selected nucleic acid or amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably, at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared.

As used herein, the term “substantially homologous” encompasses sequences that are similar to the identified sequences such that antibodies raised against peptides having the identified sequences will specifically bind to peptides possessing the “substantially homologous” amino acid sequence. In some variations, the amount of detectable antibodies induced by the homologous sequence is identical to the amount of detectable antibodies induced by the identified sequence. In other variations, the amounts of detectable antibodies induced are substantially similar, thereby providing immunogenic properties. For example, “substantially homologous” can refer to at least about 75%, preferably at least about 80%, and more preferably at least about 85% or at least about 90% identity, and even more preferably at least about 95%, more preferably at least about 97% identical, more preferably at least about 98% identical, more preferably at least about 99% identical, and even more preferably still, at least substantially or entirely 100% identical (i.e., “invariant”).

The percentage of sequence identity may be calculated over the entire length of the sequences to be compared, or may be calculated by excluding small deletions or additions which total less than about 25 percent or so of the chosen reference sequence. The reference sequence may be a subset of a larger sequence, such as a portion of a gene or flanking sequence, or a repetitive portion of a chromosome. However, in the case of sequence homology of two or more polynucleotide sequences, the reference sequence will typically comprise at least about 18-25 nucleotides, more typically at least about 26 to 35 nucleotides, and even more typically at least about 40, 50, 60, 70, 80, 90, or even 100 or so nucleotides.

When highly-homologous fragments are desired, the extent of percent identity between the two sequences will be at least about 80%, preferably at least about 85%, and more preferably about 90% or 95% or higher, as readily determined by one or more of the sequence comparison algorithms well-known to those of skill in the art, such as e.g., the FASTA program analysis described by Pearson and Lipman (1988).

As used herein, the terms “treat,” “treating,” and “treatment” refer to the administration of one or more compounds (either alone or as contained in one or more pharmaceutical compositions) after the onset of clinical symptoms of a disease state so as to reduce, or eliminate any symptom, aspect or characteristic of the disease state. Such treating need not be absolute to be deemed medically useful. As such, the terms “treatment,” “treat,” “treated,” or “treating” may refer to therapy, or to the amelioration or the reduction, in the extent or severity of disease, of one or more symptom thereof, whether before or after its development afflicts a patient.

In certain embodiments, it will be advantageous to employ one or more nucleic acid segments of the present invention in combination with an appropriate detectable marker (i.e., a “label,”), such as in the case of employing labeled polynucleotide probes in determining the presence of a given target sequence in a hybridization assay. A wide variety of appropriate indicator compounds and compositions are known in the art for labeling oligonucleotide probes, including, without limitation, fluorescent, radioactive, enzymatic or other ligands, such as avidin/biotin, etc., which are capable of being detected in a suitable assay. In particular embodiments, one may also employ one or more fluorescent labels or an enzyme tag such as urease, alkaline phosphatase or peroxidase, instead of radioactive or other environmentally less-desirable reagents. In the case of enzyme tags, colorimetric, chromogenic, or fluorigenic indicator substrates are known that can be employed to provide a method for detecting the sample that is visible to the human eye, or by analytical methods such as scintigraphy, fluorimetry, spectrophotometry, and the like, to identify specific hybridization with samples containing one or more complementary or substantially complementary nucleic acid sequences. In the case of so-called “multiplexing” assays, where two or more labeled probes are detected either simultaneously or sequentially, it may be desirable to label a first oligonucleotide probe with a first label having a first detection property or parameter (for example, an emission and/or excitation spectral maximum), which also labeled a second oligonucleotide probe with a second label having a second detection property or parameter that is different (i.e., discreet or discernable from the first label. The use of multiplexing assays, particularly in the context of genetic amplification/detection protocols are well-known to those of ordinary skill in the molecular genetic arts.

### EXAMPLES

The following examples are included to demonstrate illustrative embodiments of the invention. It should be appreciated by those of ordinary skill in the art that the techniques disclosed in these examples represent techniques discovered to function well in the practice of the invention, and thus can be considered to constitute preferred modes for its practice. However, those of ordinary skill in the art should, in light of the present disclosure appreciate that many changes can be made in the specific embodiments which are disclosed and still obtain a like or similar result without departing from the spirit and scope of the invention.

### Example 1

### ESAT-6 Detection

Fabrication and Characterization of Nanoporous Silica Thin Films.

The general methods for fabrication of nanoporous silica thin films are described in the art (see e.g., Bouamrani et al., 2010; Hu et al., 2010; Hu et al., 2011; and U.S. Patent Appl. Publ. No. 2011/0065207). Briefly, the coating silicate sol was prepared by adding 14 mL of tetraethyl orthosilicate (TEOS) into a mixture of 17 mL of ethanol, 6.5 mL of distilled water, and 0.5 mL of 6 M HCl and stirred for 2 hrs at 80° C. to form a clear silicate sol. After cooling to room temperature, 10 mL of silicate sol was added to a mixture of 1.2 g of Pluronic L121, 10 mL of ethanol, and differing amounts of polypropylene glycol (PPG). The coating solution was stirred at room temperature for 2 hr, and deposited on a Si(100) wafer by spin-coating at a spin rate of 2500 rpm for 20 sec. To increase the degree of polymerization of the silica framework in the films, and to further improve their thermal stability, the as-deposited films were heated at 80° C. for 12 hrs. The films were then calcinated at 450° C. for 5 hrs to remove the organic compound. The temperature was raised at 1° C./min.

Pluronic L121 was obtained from BASF. All other chemicals were purchased from Sigma-Aldrich (St. Louis, Mo., USA). The thickness and porosity of nanoporous silica films were characterized by variable angle spectroscopic ellipsometry (J. A. Woollam Co. M-2000DI). The thickness of the thin films and their porosities were calculated using both Cauchy and effective medium approximation (EMA) models with CompleteEASE® software (version 4.58). Ellipsometric optical quantities were detected by acquiring spectra at 55°, 60°, and 65° incidence angles, in a wavelength ranging from 300 to 1800 nm. All fabricated porous silica thin films were characterized by scanning over the entire 4-inch wafer by ellipsometry. The variation of porosity and thickness was less than 0.5%.

### Example 2

### On-Chip Fractionation and Digestion of ESAT-6

Normal human serum was obtained from Valley Biomedical (Winchester, Va., USA). Recombinant ESAT-6 was purchased from Diagnostics, Inc. (Woburn, Mass., USA). As shown in FIG. 1A, FIG. 1B, and FIG. 1C, 6 μL of protein solutions was pipetted onto the silica nanoporous film and incubated for 30 min in a humidified chamber at room temperature. The protein solution was then discarded, and 10 μL of deionized water was applied onto the silica porous film to wash away larger proteins. The washing process was then repeated three times. For enzymatic digestion, 10 μL of 0.01 mg/mL trypsin dissolved in 100 mM sodium bicarbonate was applied onto the silica nanoporous film and incubated overnight at 37° C. 10 μL of elution buffer (0.1% trifluoroacetic acid [TFA]+50% acetonitrile [ACN]) was then pipetted to extract the protein fragments. The elution buffer was then removed and stored in microcentrifuge tubes for MALDI-TOF MS analysis. To test the sensitivity of ESAT-6 detection, low concentration of ESAT-6 solution were concentrated by precipitation using ammonium sulfate. The concentrated protein pellet was dissolve in 20 μL of 100 mM sodium bicarbonate. To help dissolve protein, 16 M urea was added into the solution to reach 2 M final concentration.

MALDI-TOF MS Analysis of ESAT-6.

A matrix solution of 4 g/L of α-cyano-4-hydroxycinnamic acid (HCCA) was prepared in the solution of ACN and water (1:1, vol./vol.) which contained 0.1% TFA. 0.5 μL of each sample was spotted onto the MALDI target plate first, waiting to dry at room temperature. Next, 0.5 μL of the matrix solution was spotted onto the target plate and allowed to dry at room temperature. Mass spectra were then collected using a MALDI-TOF/TOF Analyzer (Model 4700, Applied Biosystems), in the positive reflectron mode in the 800- to 5000-Da range. Mass spectra were acquired from 5000 laser shots under 4300 laser intensity, and calibrated externally using a peptide calibration standard. Raw spectra were processed with DataExplorer® software (Applied Biosystems).

XPS (X-Ray Photoelectron Spectroscopy) Depth Profiling.

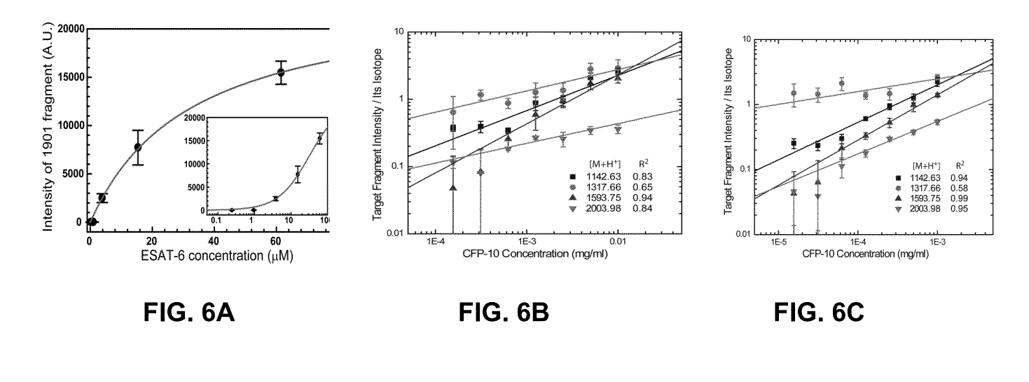

10 μM of ESAT-6 dissolved in 100 mM NaCl was incubated on 6-nm and 8-nm NSCs, and then washed with deionized water. NSCs were incubated in a vacuum chamber overnight prior to XPS measurement. PHI Quantera® XPS equipped with an Ar ion gun was used to construct concentration depth profiles. The Ar ion sputtered NSCs at accelerating voltage 3 kV in a 2×2 mm area. Because the thickness of silica film was determined by ellipsometry, the etching rate on porous silica could be calibrated by sputtering until oxygen (O1s) signal vanished. A 9-sec sputtering time interval was employed to reach a 5.25-nm depth spacing with a 35 nm/min Ar ion etching rate. Nitrogen (Nis) spectra were observed to identify the amount of ESAT-6 trapped at different depths (see e.g., FIG. 6A, FIG. 6B, and FIG. 6C).

Pre-concentration of ESAT-6.

1 mL of each concentration of ESAT-6 in urine was mixed with 0.4 g of ammonium sulphate. The solution was vortexed for 30 min; precipitated proteins were collected by bench top centrifugation, and the supernatant was then removed. 20 μL of 100 mM ammonium bicarbonate and 2 μL of 16 M urea were used to dissolve the precipitated proteins. The concentrated protein solution was then directly applied to NSCs for on-chip fractionation.

### Indirect ELISA of ESAT-6

### Comparative Example

Recombinant ESAT-6, mouse monoclonal antibody against ESAT-6, and indirect ELISA kits were all obtained from Abcam, Inc. To test the sensitivity of indirect ELISA of ESAT-6 in different human bodily fluids, a series of concentration of recombinant ESAT-6 dissolved in 1×PBS buffer, urine, and 5% human serum in 1×PBS buffer was prepared. The manufacturer's standard protocol for indirect ELISA was followed: the antigens were first coated on microplates by incubation at 4° C. overnight. The remaining protein-binding sites were blocked by 5% serum in 1×PBS buffer. The antigen was incubated with primary antibody, and then conjugated with secondary antibody. 3,3′,5,5′-tetramethylbenzidine (TMB) was used as a detection reagent.

### Example 3

### ESAT-6/CFP-10 Detection Analyses

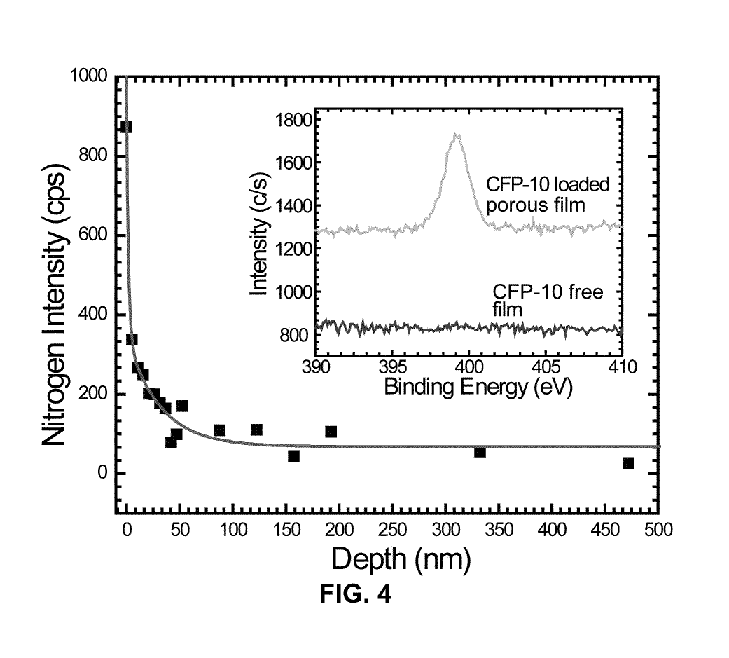

An exemplary detection procedure in accordance with one aspect of the present invention is illustrated in FIG. 1A. The biological samples were applied to a silicone gasket culture well (3-mm diameter and 1-mm height) attached on top of the nanoporous silica film. The fractionation process was completed after serial washes as described in Examples 1 and 2. Because the silica films were fixed on the flat substrate, the fractionation process was easily applied without any inconvenient washing procedure, such as sedimentation steps usually required in particle-based systems. The relatively small size of ESAT-6 (MW=10 kDa) allowed it to be captured by the silica nanopores. To identify ESAT-6 protein from biological fluids with MALDI-TOF MS, a mass fingerprinting must first be established. Because smaller protein or peptides species provide higher signals and resolution in MS and full-length ESAT-6 (10 kDa) is relatively large for MALDI-TOF MS under linear mode (FIG. 4), the proteolytic enzyme, trypsin, was used to cleave full length ESAT-6 into smaller fragments. In contrast to conventional digestion processes, which are usually conducted in bulk solution, the digestive enzymes in this case were applied directly onto the nanoporous silica film and they interacted with protein inside the nanopores. Several advantages of this novel nanobiocatalysis process over conventional solution-based digestion have been reported, including higher efficiency and better stability (Kim et al., 2010; Qiao et al., 2008; and Savino et al., 2011). In addition, the on-chip digestion protocol eliminated several additional processes, including protein extraction from the silica nanopore and buffer exchange for enzymatic digestion can further simplify the operation procedures.

After 10 hrs' incubation, the proteins retained inside the silica pores were trypsin-digested into smaller fragments, which were then extracted with elution buffer. The fingerprinting spectrum of ESAT-6 showed a strong signal from two major fragments corresponding to mass [M+H]⁺1900.9511 Da and 1907.9246 Da. LC-MS was used to confirm that these two fragments did indeed originate from proteolysis of ESAT-6 protein (1900.9511 m/z, amino acid sequence: WDATATELNNALQNLAR [SEQ ID NO:1]; 1907.9246 m/z, amino acid sequence: LAAAWGGSGSEAYQGVQQK, [SEQ ID NO:2]). Although these fragments did not represent the only degradation products from trypsin digestion of ESAT-6, these two peptides presented the strongest mass peaks due to their specific physiochemical properties. The results suggested that these two fragments were excellent markers for ESAT-6.

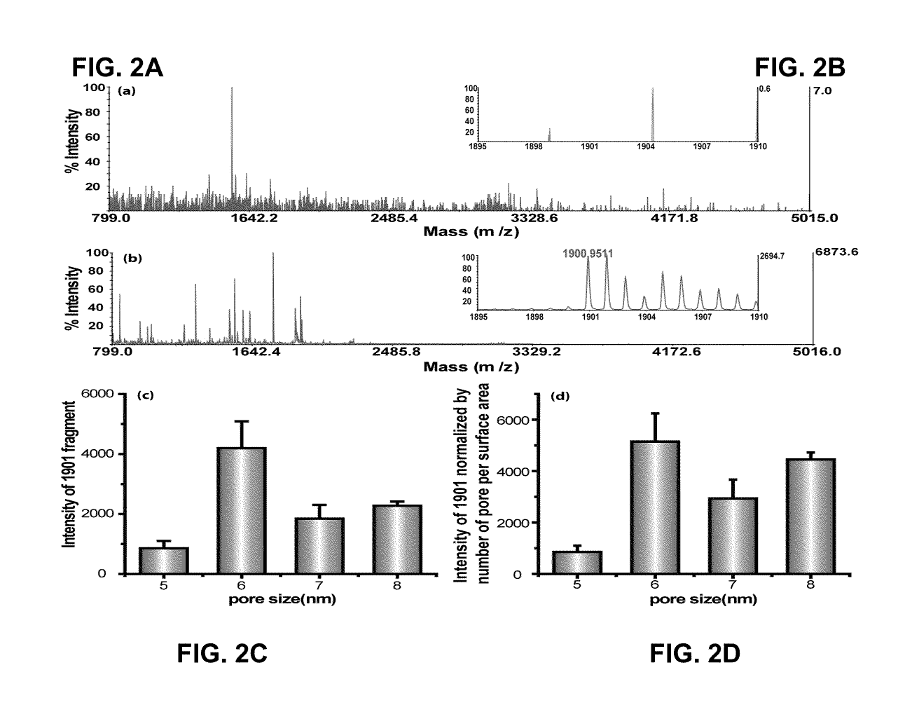

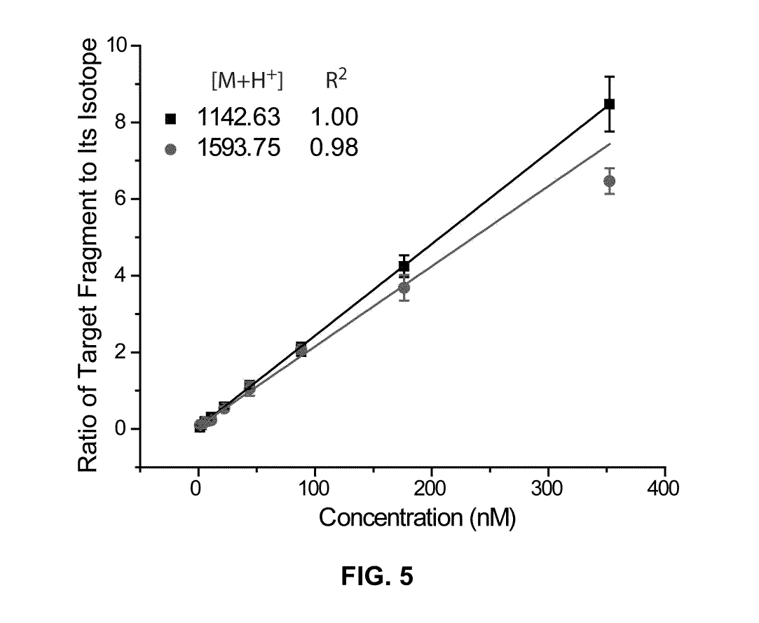

To examine the fractionation capability of the nanoporous silica film, the recombinant ESAT-6 was mixed with human serum and then treated with or without on-chip separation, followed by digestion with trypsin, as shown in FIG. 2A, FIG. 2B, FIG. 2C, and FIG. 2D. Without fractionation, the signal of the major ESAT-6 fragment, 1900.9511[M+H]⁺ at a high concentration of 80 μM of ESAT-6, was obscured (FIG. 2A). With on-chip fractionation, the detection of ESAT-6 fragment from the same complex biological fluid was significantly improved (FIG. 2B). Human serum without ESAT-6 was also fractionated and digested on nanoporous silica film as a control. No fragments were identified in the range from 1895 to 1910 Da (FIG. 5). These results indicate that the most abundant species in human serum that may overlap with the ESAT-6 fragments were removed by the fractionation process, which enabled the enrichment and quantification of ESAT-6 from human serum using MS.

Optimization of Pore Size.

Concentration gradient is a major driving force of protein diffusion into nanopores. Interactions between proteins and the silica surface, especially electrostatic interactions, can play a significant role in protein absorption, and can impact the isolation process (Deere et al., 2002; Katiyar et al., 2005). In addition, the architecture of the nanoporous silica films and the shape and size of target proteins can influence the capturing effects. Previous studies have indicated that the effect of pore blockage by adsorbed proteins is eliminated when the pore diameter is twice as large as the hydrodynamic size of protein (Vinu et al., 2004). Large pores, however, may also enrich large and abundant peptides that can reduce the sensitivity of target LMW molecule in MALDI-TOF MS. Furthermore, during the on-chip digestion process, trypsin interacts with either captured protein within the pore, given sufficient space for proper operation, or proteins that have been released back into solution, indicating that pore size will also influence on-chip digestion efficiency.

In this context, various pore sizes were tested for ESAT-6 enrichment by adding a swelling agent, polypropylene glycol (PPG), which interacts with the hydrophobic domain to expand the micelle template during nanoporous silica film fabrications (Hu et al., 2010; Sorensen et al., 2008). Pluronic triblock copolymer L121 mixed with PPG at 0, 25, 50 and 100 wt % resulted in approximately 5-, 6-, 7- and 8-nm average pore diameters, respectively. After performing the fractionation process and on-chip digestion using the nanoporous silica films with different average pore sizes, the highest intensity of 1900.9511 [M+H] Da fragment was observed in the 6-nm average diameter pores (FIG. 2C). Despite the larger average pore size having less steric obstructions for protein diffusion, the higher enrichment efficiency of a 6-nm pore could be attributed to the higher number of pores per unit surface area.

The porosity of nanoporous silica film was measured by ellipsometry as described in Example 1. The normalized number of pores per unit surface area was evaluated by porosity, as reported in Table 1. There were 1.6 times as many 6-nm pores per unit surface area in the nanoporous silica thin-films than there were in films with 8-nm pores. After normalizing of the 1900.9511 [M+H] fragment peaks to the number of pores per unit surface area, no major distinction was observed between 6, 7 and 8 nm pore sizes (FIG. 2D). The 5-nm pores, however, showed only one-fifth the intensity of 6-nm pores. This may attributable to the geometry of ESAT-6 that exhibits a rod-like structure with dimensions of 1.5×6 nm (see Renshaw et al., 2005). The long backbone (rod-like) of the ESAT-6 molecule produces anisotropic diffusion that may reduce diffusion rates inside silica nanopores; especially in the smaller 5 nm pores, where the ESAT-6 and the pores are similar in size (Eichmann et al., 2011). In addition, 5-nm pores are also similar in size to trypsin, a globular-like protein with a 4.5 nm diameter (Leiros et al., 2004). The low diffusion rate of trypsin in these smaller pores would reduce the digestion efficiency inside the nanopore.

TABLE 1
PROPERTIES OF EXEMPLARY FABRICATED
NANOPOROUS SILICA CHIPS

| | Pore | | Pore Cross | Normalized |
|---|---|---|---|---|
| | Size | Porosity | Sectional Area | Number |
| | (nm) | (%) | (nm²) | of Pores/Area* |
| L121 | 5 | 52 | 19.63 | 1 |
| L121 + 25% PPG | 6 | 61 | 28.27 | 0.81 |
| L121 + 50% PPG | 7 | 64 | 38.485 | 0.63 |
| L121 + 100% PPG | 8 | 68 | 50.27 | 0.51 |

*Number of pores per area calculated based on porosity and pore size.

Adapting Nanopore Morphology Influences CFP-10 Enrichment.

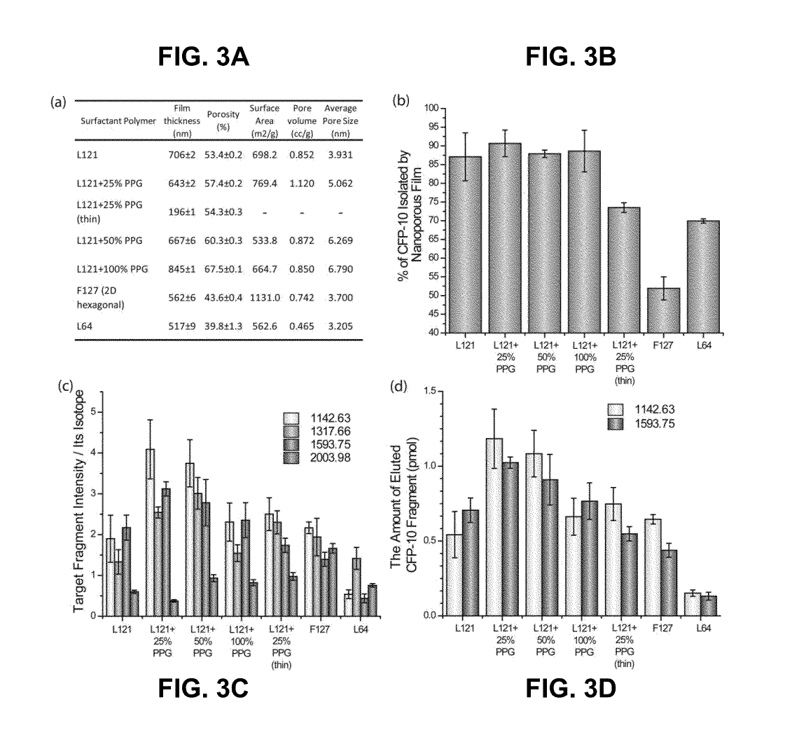

Different design parameters (e.g., pore size and shape, chemistry, porosity, etc.) dictate the “landscape” and ultimately the peptidic fingerprint of samples processed by on-chip fractionation. To determine the optimal morphology for CFP-10 isolation, the pore morphology was adjusted by using different copolymers and the swelling agent, polypropylene glycol (PPG), which interacts with the hydrophobic domain of polymers to expand the micelle template during NPS film fabrications. Mixing various compositions of the pluronic triblock copolymers F127, L64, and L121mixed with PPG at 0, 25, 50 and 100% weight of the copolymers yielded a number of film thickness, porosity, surface area, pore volume and sizes (FIG. 3A).

The isolation efficiency of recombinant CFP-10 was first analyzed as a function of NPS configurations. 0.05 mg/mL of CFP-10 dissolved in PBS buffer was incubated in the silicon gasket well (3-mm diameter) pasted on NPS surface. After extensive washes, the amount of CFP-10 remaining in the wash solution was quantified using indirect ELISA, and the percentage of CFP-10 retained in the morphologically distinct nanopores was calculated (FIG. 3B). Significantly lower isolation efficiency was observed when the highly-ordered nanoporous film (F127) was used for fractionation, compared to the use of non-ordered nanoporous films (L121), although the average nanopore sizes of both films were comparable (pore size of F127: 3.7 nm vs. L121: 3.9 nm). It was previously shown that the F127 film consists of 2-dimensional (2-D) hexagonal and closely-packed nanopores that are perpendicular to the film's surface. In contrast, L121 or L121+PPG films are composed of non-ordered, or worm-like, nanoporous structures. These results suggested that a non-ordered nanopore structure was more conducive to isolating CFP-10.

Pore size also strongly influences the fractionation efficiency. Among the non-ordered nanoporous films, L64 film with 3.2-nm average nanopore diameter displays lower isolation efficiency than other films (L121 & L121+PPG). Moreover, the films consisting of nanopore size above 3.9 nm showed similar CFP-10 isolation efficiencies that were irrespective of the addition of PPG (L121 and L121+PPG). This result suggested that the rod-like CFP-10 with dimensions of 1.5 nm×6 nm was not significantly excluded by nanopores larger than 3.9 nm.

Modifying the nanopore film thickness, without interfering with pore morphology, also influences the efficiency of sample peptide retention and enrichment. The thickness can be manipulated by diluting the coating sol, which is the silicate sol mixed with polymer in ethanol, or by controlling the rotational speed of the spin coater. L121+25% PPG films were synthesized in two varieties: a 643-nm (“thick”) version and a 196-nm (“thin”) version. It was observed that the “thick” film captured more CFP-10 peptides. To better understand this phenomenon, the amount of CFP-10 penetrating into the nanoporous film was measured using X-ray photoelectron spectroscopy (XPS). As presented in FIG. 4, the concentration of CFP-10 declined exponentially as a function of nanoporous film thickness and the majority of peptide accumulated within the top 100-nm layer. It was reasoned that it was not for lack of sufficient peptide holding space in the 196 nm films, but for the fact that the 643 nm thicker films provided additional reservoirs needed for the capillary-guided water flow and filling action that enhances the diffusion of CFP-10 within nanopore structures. Of all the nanopore configurations tested, the one resulting in an isolation efficiency up to 90% (36 ng of CFP-10) exhibited the following parameters: L121+25% PPG, 632-nm thickness, 7-mm² surface area, and a 30-min incubation (FIG. 3B).

Adjusting the concentration of PPG not only affects pore size, but doing so also changes the structure's porosity, defined as the fraction of void spaces in the film. In FIG. 3B, comparable CFP-10 isolation efficiencies were observed when the L121 and L121+PPG films were used. However, other parameters were also considered that could singly, or collectively, improve the peptide enrichment and detection procedure, including the likely exclusion of abundant protein species in the sample, the efficiency of trypsin digestion, and sample elution. To test this hypothesis, recombinant CFP-10 was “spiked” into sterile MTB culture medium, the samples were processed on nanopore films of distinct characteristics, detected through MS, and then the MS signals of CFP-10 fragments were compared (FIG. 3C). To minimize the variation caused by the intrinsic fluctuations of MALDI-TOF MS, each extracted sample was spiked with isotopic peptides in known quantities to serve as internal standards. These isotopic peptides were synthesized by digested CFP-10 in ¹⁸O-enriched-water (H₂¹⁸O), leading to their shift in mass by 4 Da without changing any other physical properties. Each MS signal shown in FIG. 3C was normalized by its own isotopic fragments. Although adjusting the pore size and porosity of L121 with PPG did not alter the amount of isolated CFP-10 (FIG. 3B), its impact became much more evident when the MS data were examined (FIG. 3C). Here, addition of PPG did have a positive effect on the detections of CFP-10 fragments, with the highest MS signals observed when the sample was processed on the L121+25% PPG nanoporous film. This increase tapers down and plateaus with further addition of PPG (100%). One possible reason for these observations is that the small pore size of L121 without PPG (avg. pore size: 3.9 nm) hinders the diffusion of globule-like trypsin (4 nm diameter), preventing interactions between trypsin and CFP-10 retained inside the nanotraps. With pore sizes beyond 5 nm, the effect of PPG on trypsin digestion was once again minimal to none (compare L121+25% PPG and L121+50% PPG to L121). Additionally, the larger pores retain more of the abundant proteins present in the sample, leading to a MS signal reduction of CFP-10 fragments. Indeed, a significant decrease in MS signal intensity was observed when the L121+100% PPG film (avg. pore size: 6.8 nm) was used.

Determining the Amount of CFP-10 from MTB Cultures.

To quantify the absolute amount of CFP-10 fragments by their isotopic fragments, a standard curve of the signal ratio of each monoisotopic and ¹⁸O-labeled fragment was established (FIG. 5). The isotopic fragments shifted by 4 Da to partially overlap with the mono-isotopic fragments. The fragments with 1142.63 and 1593.75([M+H]⁺)⁺) in MALDI-TOF MS showed good linear regression between MS signal intensity and fragment quantity below 400 nM (FIG. 5, R2=1.00 and 0.98, respectively), whereas the fragments of 1317.66 and 2003.98 ([M+H]⁺)⁺) demonstrated poor linear regression (R2=0.86 and 0.50, respectively). Based on standard curves for the fragments with 1142.63 and 1593.75 ([M+H]⁺) the amount of CFP-10 was quantified after on-chip sample processing on different nanopore films. Similar to earlier results, CFP-10 processed on L121+25% PPG resulted in the highest yield at as 1.2 pmol (FIG. 3D).

To test the sensitivity of the assay, and determine its minimum threshold of detection, recombinant CFP-10 was titrated in sterile MTB culture medium, and each sample was processed on the L121+25% PPG film and through MS. The MS signals of four major fragments, each normalized to its own isotope, are depicted in FIG. 6B, plotted as signal intensity versus CFP-10 concentration in a log-log plot. Linear regression ranged from acceptable to good. Based on these results, this assay could detect CFP-10 in culture medium at a remarkably low concentration of 13.4 nM. The limit of detection was further improved (to 1.3 nM) by concentrating CFP-10 ten-fold by ammonium sulfate precipitation of the culture media before on-chip processing (FIG. 6C).

TABLE 2
INTER-DAY ACCURACY AND REPRODUCIBILITY OF CFP-10
ON-CHIP FRACTIONATION-MS ANALYSIS

| Concentration | | Mean | Standard | Precision | Accuracy |
|---|---|---|---|---|---|
| (μg/mL) | Fragments | (μg/mL) | Deviation | (% CV) | (% RE) |
| 1000 | 1317.664 | 1.7127 | 0.9940 | 58.03% | 71.27% |
| | 2003.978 | 0.0559 | 0.0351 | 62.66% | 94.41% |
| 125 | 1317.664 | 0.1361 | 0.0998 | 73.33% | 8.91% |
| | 2003.978 | 0.0559 | 0.0351 | 62.66% | 55.24% |
| 15.625 | 1317.664 | 0.4283 | 0.4536 | 105.90% | 2641.13% |
| | 2003.978 | 0.0031 | 0.0030 | 97.31% | 80.20% |

To access the inter-day and intra-day variability of the combined on-chip fractionation-MS analysis, recombinant CFP-10 was spiked at three different and defined concentrations, in replicate samples, into sterile culture media. The fragments with 1142.63 and 1593.75([M+H]⁺) displayed higher MS signals (see FIG. 1A, FIG. 1B, and FIG. 1C), better linear regression with respect to their isotopes, and better quantification accuracy (% RE, relative error) and precision (% coefficient of variation, CV) compared to the fragments with 1317.66 and 2003.98 ([M+H]⁺)⁺) (see Table 2 and Table 3). At 100 nM concentration, the mean calculated concentrations remained within 10% of the actual values (% RE) and did not exceed 30% of the % CV. At lower concentrations (e.g., 1.3 nM), the accuracy of quantification decreased to 73%. The qualitative identification of CFP-10 remained very precise even at only 1.3 nM. Strong MS signals were detected for fragments with 1142.63 and 1593.75([M+H]⁺)⁺) in all of the samples (n=11) and the fragments with 1317.66 and 2003.98 ([M+H]⁺)⁺) in 63% and 72% of the samples.

TABLE 3
INTRA-DAY ACCURACY AND REPRODUCIBILITY OF CFP-10
ON-CHIP FRACTIONATION-MS ANALYSIS

| Con- | | | | | |
|---|---|---|---|---|---|
| centration | | Mean | Standard | Precision | Accuracy |
| (μg/mL) | Fragments | (μg/mL) | Deviation | (% CV) | (% RE) |
| 1000 | 1317.664 | 1.2903 | 0.6716 | 52.05% | 29.03% |
| | 2003.978 | 0.0732 | 0.0412 | 56.32% | 92.68% |
| 125 | 1317.664 | 146.5446 | 78.1665 | 53.34% | 17.24% |
| | 2003.978 | 0.0732 | 0.0412 | 56.32% | 41.44% |
| 15.625 | 1317.664 | 0.4283 | 0.4536 | 105.90% | 2641.13% |
| | 2003.978 | 0.0029 | 0.0033 | 112.94% | 81.30% |

Differentiating MTB Based on its CFP-10 Signatures in Clinical Isolates.

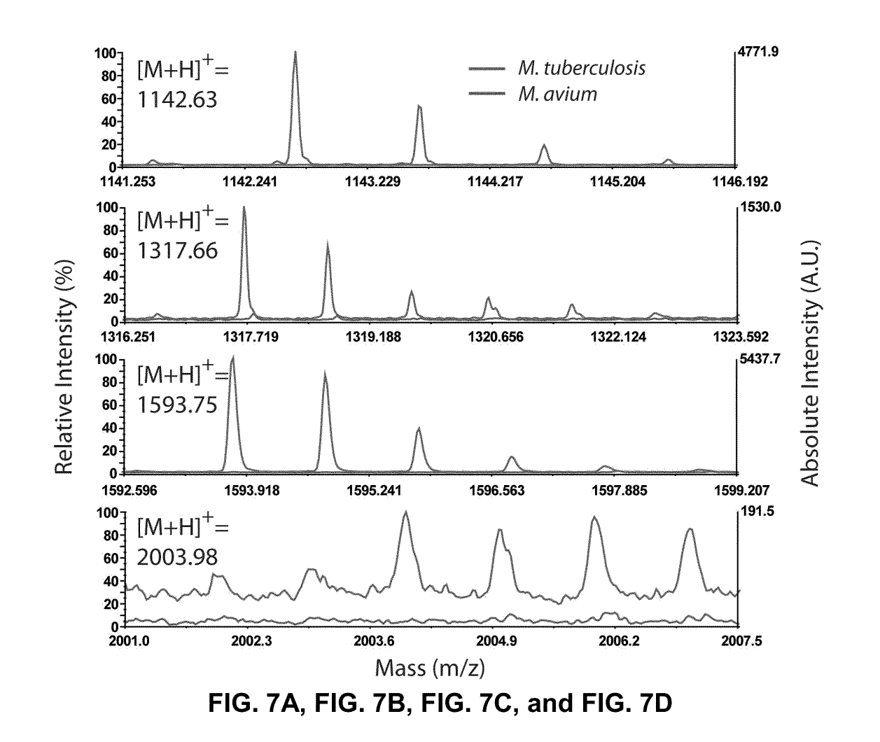

To address specificity of the on-chip fractionation-MS technology, the expression of CFP-10 from MTB grown in culture medium was investigated (FIG. 7). The non-TB Mycobacterium (NTM), Mycobacterium avium (M. avium) lacks the CFP-10 gene, and therefore serves as a negative control. To mimic conditions one may find in early-disease diagnosis (i.e, low secretion of CFP-10 in the culture supernatant), the same ammonium sulfate concentration protocol was performed prior to on-chip fractionation and MS analysis. Indeed, strong MS signals for the all fragments were observed in the supernatant of MTB culture, but not in the M. avium culture (FIG. 7).

Sensitivity of Assay and Pre-Concentration of ESAT-6.

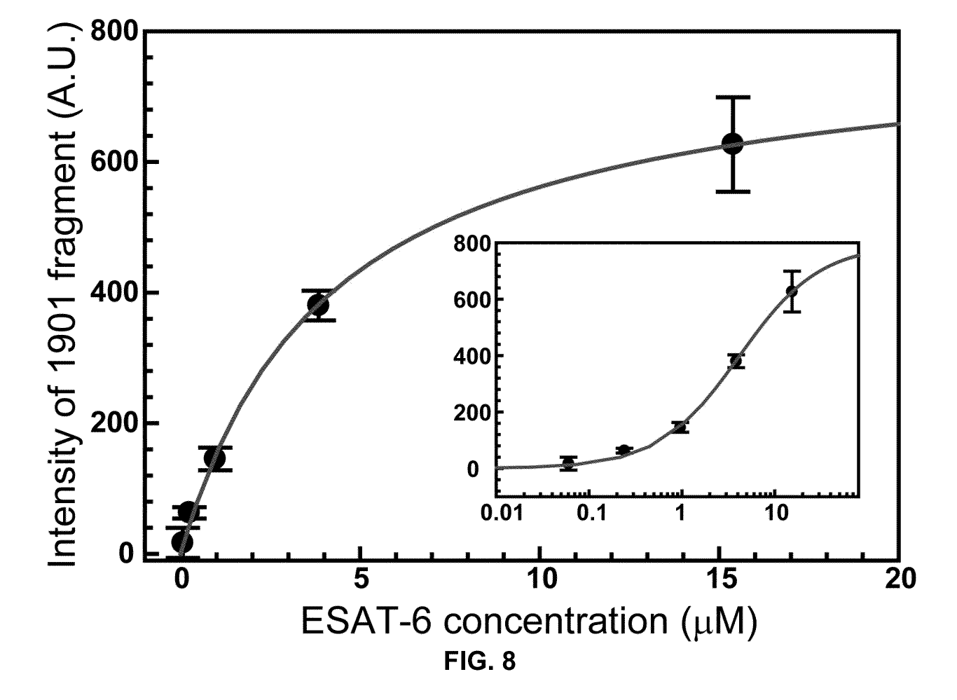

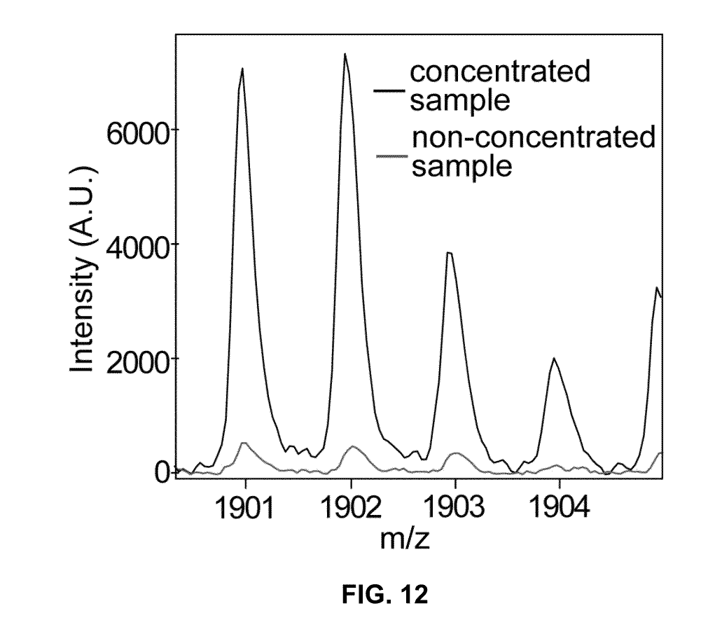

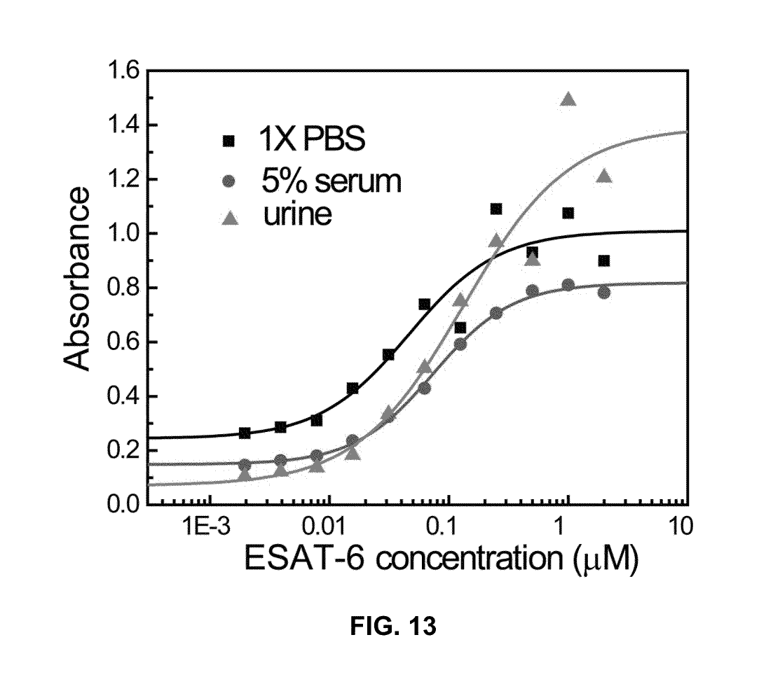

To test the sensitivity of the current assay, on-chip fractionation and digestion process was applied to human serum and urine samples mixed with different concentrations of ESAT-6. The intensities of the 1901 Da fragment mass peak were measured by MALDI-TOF MS at different concentrations, as shown in FIG. 8 and FIG. 13. 60 nM of ESAT-6 dissolved in either human serum (5%) or urine was still detectable using the nanopore-based assay described herein. To enhance the sensitivity of the assay, proteins within the patient sample were concentrated by precipitation before being applied onto sample on the surface of the nanoporous silica thin-film. As shown in FIG. 12, standard protein precipitation procedure increased ESAT-6 concentration in urine samples and enhanced the detection signal.

### ELISA of ESAT-6

### Comparative Example

Although enzyme-linked immunosorbent assay (ELISA) is often used to detect particular antigens in biological fluids, highly sensitive ELISAs (either sandwich or competitive ELISA), are not yet available. The indirect ELISA suffers from low sensitivity, especially in serum samples, because the analyte needs to compete with other abundant protein in biological fluids. The indirect ELISA was performed to detect ESAT-6 mixed in different biological fluids. As shown in FIG. 13, in the biological buffer without any other proteins, ESAT-6 signals were observed at 2 nM concentration. However, in human serum sample, ESAT-6 signals were not observed below micro-molar concentration. In contrast, the nanopore-based assay described herein sufficiently removed abundant proteins from complex body fluids, and thus enhanced the sensitivity of ESAT-6 detection in such body fluids.

This Example illustrates a novel sample pretreatment protocol for MALDI-TOF MS using nanoporous silica films to fractionate and enrich specific biomarkers, such as ESAT-6-specific peptides. These on-chip fractionation and digestion protocols were established to provide a facile and efficient method for TB screening and diagnosis. The silica nanopores of the disclosed films were fabricated to capture the specific biomarker peptides of interest (here, ESAT-6), to exclude the high-abundance proteins present in the sample that would otherwise obscure the signal of the target molecule(s) during MS analysis, and to boost the level of detection of the specific biomarker peptide of interest. The experimental results showed that by specifically engineering the pore size, porosity, and surface chemistry of the nanoporous silica films, the ability of the film to capture particular biomarker molecules of interest could be precisely controlled.

The method described herein is applicable not only to the detection of the test biomarkers illustrated herein, but may also be extended to the identification of a range of other proteins, peptides, and specific biomarkers of interest.

### Example 4

### Simultaneous Detection of ESAT-6 and CFP-10

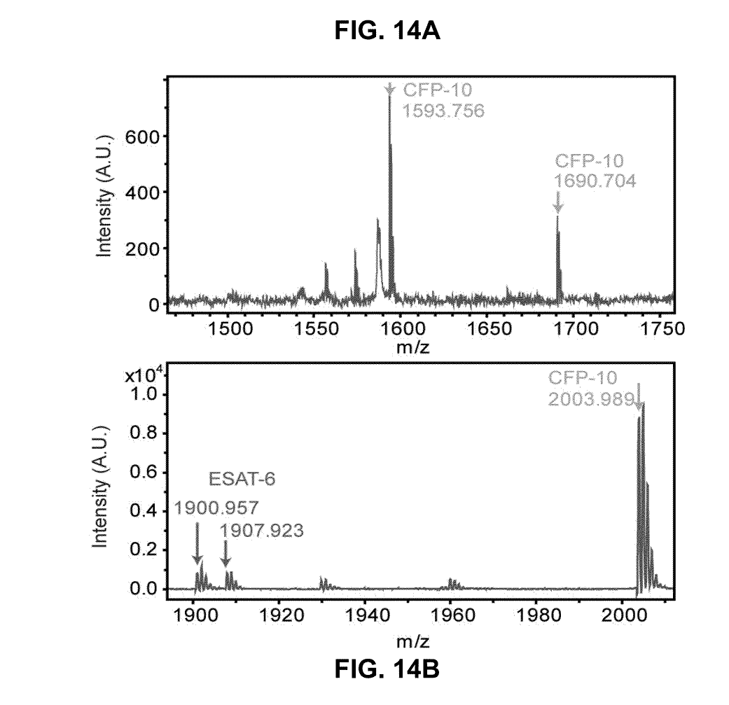

To improve the sensitivity and specificity for TB diagnosis, both ESAT-6 and CFP-10 were detected simultaneously from human body fluids. First, the finger printing spectra of ESAT-6 and CFP-10 were established (FIG. 14). Two major digestion fragments of ESAT-6 (molecular weights 1900.9511 and 1907.9246) were observed in the spectrum. Comparing to ESAT-6 fragments, more CFP-10 fragments were observed in MALDI-TOF MS, including 2003.9781, 1668.7170, 1593.7503, 1142.6276, and 908.4584 (Table 4). The most significant CFP-10 fragment (2003.9781) showed 10-fold higher in the intensity than the ESAT-6 fragment, which improved the sensitivity of this assay.

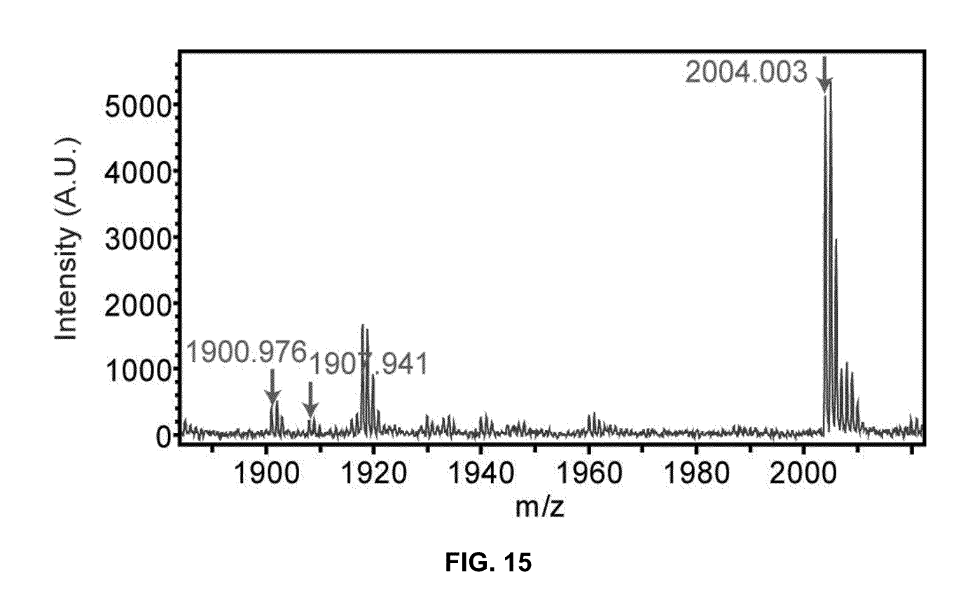

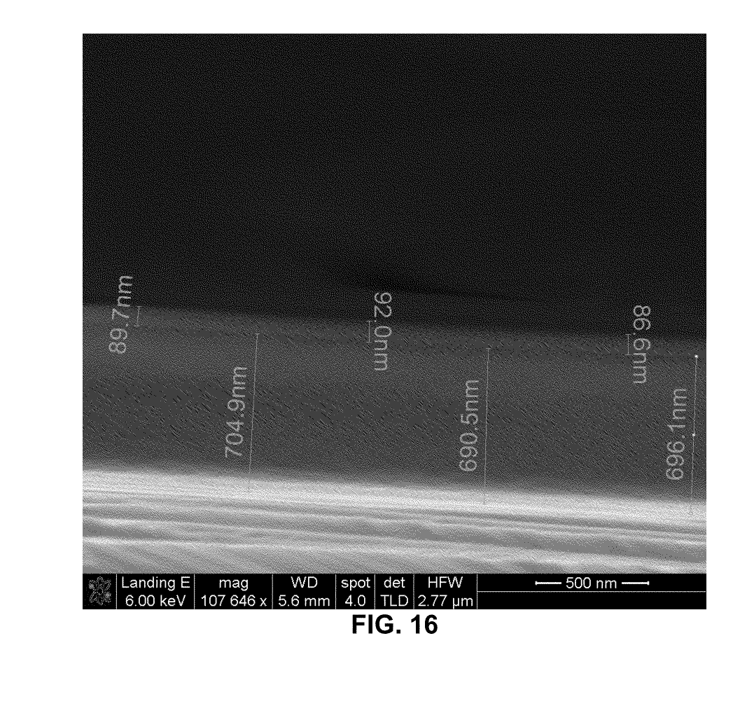

The human serum containing both 80 μM CFP-10 and 80 μM ESAT-6 antigens were fractionated with nanoporous silica films having a 6-nm average pore diameter, and then digested with trypsin. The standard protocol was then applied to process this sample as described above. MALDI-TOF MS was then used to detect the antigen fragments in the eluted samples. The mass spectra showed that CFP-10 and ESAT-6 fragments were simultaneously observed (FIG. 15). CFP-10 showed higher intensity than ESAT-6. To improve the detection limit of nanoporous silica films in human serum, a 30-kDa cut-off ultracentrifugal filter was adopted for pre-concentration purposes. Then the pre-concentrated human serum with both CFP-10 and ESAT-6 was loaded and processed on nanoporous silica film (single-layer or dual-layer). As shown in FIG. 16, the detection limit of CFP-10 can reach as low as 100 nM in human serum after treated by both single-layer or dual-layer nanoporous silica film. Dual-layer nanoporous silica film showed less non-specific peaks than the single-layer one. Based on this result, it can be concluded that dual-layer nanoporous silica film increased the capture efficiency of target proteins compared to the single-layer one. After optimization, CFP-10 and ESAT-6 could be detected at concentrations less than 10 nM in human body fluid by coupling ultracentrifugal filtration with subsequent on-chip fractionation. Therefore, the assay described herein is useful to detect two or more antigens released from one or more infectious diseases. It offers significant improvements in sensitivity and specificity compared to existing assays, and has proven suitable for identifying multiple antigens/diseases in a single test.

TABLE 4
SEQUENCES AND MOLECULAR WEIGHTS OF CFP-10 AND
ESAT-6 DIGESTED FRAGMENTS

| | | Amino Acid Sequence of |
|---|---|---|
| Peptide | [M + H]⁺ | Resulting Fragments |
| CFP-10 | 2003.9781 | TQIDQVESTAGSLQGQWR |
| | | (SEQ ID NO: 3) |
| | 1668.7170 | ADEEQQQALSSQMGF |
| | | (SEQ ID NO: 4) |
| | 1593.7503 | TDAATLAQEAGNFER |
| | | (SEQ ID NO: 5) |
| | 1142.6276 | GAAGTAAQAAVVR |
| | | (SEQ ID NO: 6) |
| | 908.4584 | QAGVQYSR |
| | | (SEQ ID NO: 7) |
| ESAT-6 | 1907.9246 | LAAAWGGSGSEAYQGVQQK |
| | | (SEQ ID NO: 2) |
| | 1900.9511 | WDATATELNNALQNLAR |
| | | (SEQ ID NO: 1) |

### Example 5

### Fabrication of Dual-Layer Nanoporous Film

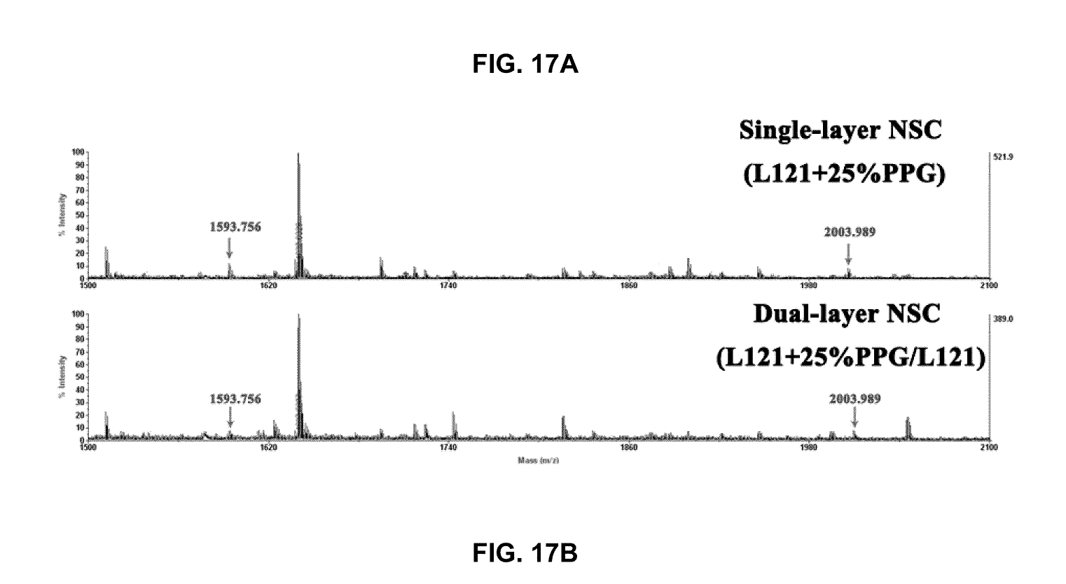

The dual layers of porous silica were fabricated using layer-by-layer coating protocol. For example, as shown in FIG. 17, the double-layer silica film containing 5- and 4-nm average pore diameters on the top and bottom layers, respectively, was fabricated by serially coating Pluronic L121 and Pluronic L121+25% PPG silicate sol on silicon wafers, according to the following protocol:

1. The different coating sols were prepared by mixing silicate sol with different type of Pluronic polymer. For 4 nm size pore, 10 mL of silicate sol was adding to the mixture of 1.2 g of Pluronic L121, and 5 mL of ethanol. For 5-nm size pore, 10 mL of silicate sol was added to a mixture of 1.2 g of Pluronic L121, 30 mL of ethanol, and 0.3 g of polypropylene glycol (PPG).

2. The coating solution was stirred at room temperature for 2 hr. The L121 coating sol was then deposited on a Si(100) wafer by spin coating at the spin rate of 500 rpm for 5 sec followed by spin coating at the spin rate of 2000 rpm for 20 sec. To increase the degree of polymerization of the silica framework in the films and to further improve their thermal stability, the as-deposited films were heated at 80° C. for 12 hrs. The films were calcinated firstly at 175° C. for 3 hrs and secondly at 450° C. for 5 hrs to remove the organic compound.

3. After the formation of first layer, the second layers were fabricated by depositing L121+25% PPG coating sol on the top of 4 nm pore size layer by spin coating at a spin rate of 500 rpm for 5 sec followed by spin coating at a spin rate of 2000 rpm for 20 sec. The films were first baked at 80° C. for 12 hrs, at 175° C. for 3 hrs, and then calcinated at 450° C. for 5 hrs to remove the organic compound.

As noted above, multilayer silica films can also be fabricated by repeating this layer-by-layer coating process in a stepwise fashion, until the desired number of layers are obtained.

### REFERENCES

The following references, to the extent that they provide exemplary procedural or other details supplementary to those set forth herein, are specifically incorporated herein in their entirety by express reference thereto.
U.S. Patent Appl. Publ. No. 2011/0065201 entitled “Nanostructure material with metallic oxide and method for detecting phosphorus-containing compounds,” to Yen et al. (priority to Sep. 15, 2009).
U.S. Patent Appl. Publ. No. 2011/0065207, entitled “Combinatorial multidomain mesoporous chips and a method for fractionation, stabilization, and storage of biomolecules,” to Ferrari et al. (priority to Jul. 20, 2009).
U.S. Patent Appl. Publ. No. 2012/292255, entitled “Filtration and use of metal nanoparticles as non-optical tags in chemical-, bio-chemical sensors and micro-electromechanical devices,” to Nikoobakht (priority to May 16, 2011).
PCT Intl. Pat. Appl. Publ. No. WO 2007/120248, entitled “Nanoporous substrates for analytical methods” to Ferrari et al. (priority to Dec. 20, 2005).
BOUAMRANI, A et al., “Mesoporous silica chips for selective enrichment and stabilization of low molecular weight proteome,” Proteomics, 10(3):496-505 (February 2010).
CHUN, A L, “Nanoparticles offer hope for TB detection,” Nat. Nanotechnol., 4(11):698-699 (November 2009).
DEERE, J et al., “Mechanistic and structural features of protein adsorption onto mesoporous silicates,” J. Phys. Chem. B. 106(29):7340-7347 (June 2002).
DOHERTY, T M et al., “Immune responses to the Mycobacterium tuberculosis-specific antigen ESAT-6 signal subclinical infection among contacts of tuberculosis patients,” J. Clin. Microbiol., 40(2):704-706 (February 2002).
DUNLAP, N E et al., “Diagnostic standards and classification of tuberculosis in adults and children,” Am. J. Resp. Crit. Care, 161(4):1376-1395 (April 2000).
FLORES, L L et al., “Systematic review and meta-analysis of antigen detection tests for the diagnosis of tuberculosis,” Clin. Vacc. Immunol., 18(10):1616-1627 (August 2011).
HOFF, S T et al., “Evaluation of Mycobacterium tuberculosis-specific antibody responses in populations with different levels of exposure from Tanzania, Ethiopia, Brazil, and Denmark,” Clin. Infect. Dis., 45(5):575-582 (July 2007).
HU, Y et al., “Microfluidic enrichment of small proteins from complex biological mixture on nanoporous silica chip,” Biomicrofluidics, 5(1):013410 (March 2011).
HU, Y et al., “Surface engineering on mesoporous silica chips for enriching low molecular weight phosphorylated proteins,” Nanoscale, 3(2):421-428 (February 2011).
HU, Y et al., “Tailoring of the nanotexture of mesoporous silica films and their functionalized derivatives for selectively harvesting low molecular weight protein,” ACS Nano., 4(1):439-451 (January 2010).
HUSSAIN, M M et al., “Unmodified gold nanoparticles for direct and rapid detection of Mycobacterium tuberculosis complex,” Clin. Biochem., 46(7-8):633-637 (May 2013).
JIN, A. et al. “A rapid and efficient single-cell manipulation method for screening antigen-specific antibody-secreting cells from human peripheral blood,” Nat. Med., 15:1088-1092 (September 2009).
KASHYAP, R S et al., “Diagnostic value of early secreted antigenic target-6 for the diagnosis of tuberculous meningitis patients,” Infection, 37(6):508-513 (December 2009).
KATIYAR, A et al., “Protein adsorption on the mesoporous molecular sieve silicate SBA-15: effects of pH and pore size,” J. Chromatogr. A, 1069(1):119-126 (March 2005).
KIM, J et al., “Nanobiocatalysis for protein digestion in proteomic analysis,” Proteomics, 10(4):687-699 (February 2010).
LASERSON, K F et al., “Improved sensitivity of sputum smear microscopy after processing specimens with C₁₈-Carboxypropylbetaine to detect acid-fast bacilli: a study of United States-bound immigrants from Vietnam,” J. Clin. Microbiol., 43(7):3460-3462 (July 2005).
LEIROS, H K et al., “Trypsin specificity as elucidated by LIE calculations, X-ray structures, and association constant measurements,” Protein Sci., 13(4):1056-1070 (April 2004).
NGUYEN, Y H et al., “Rapid identification and drug susceptibility screening of ESAT-6 secreting Mycobacteria by a NanoELIwell assay,” Sci. Reports, 2:635 DOI:10.1038/srep00635 (September 2012).
QIAO, L et al., “A nanoporous reactor for efficient proteolysis,” Chem-Eur. J., 14(1):151-157 (October 2007).
RAVN, P et al., “Prospective evaluation of a whole-blood test using Mycobacterium tuberculosis-specific antigens ESAT-6 and CFP-10 for diagnosis of active tuberculosis,” Clin. Diagn. Lab. Immunol., 12(4):491-496 (April 2005).
RAWLINGS, N D et al., “MEROPS: the peptidase database,” Nucl. Acids Res., 38:D227-D233 (January 2010).
RENSHAW, P S et al., “Structure and function of the complex formed by the tuberculosis virulence factors CFP-10 and ESAT-6,” EMBO J., 24(14):2491-2498 (July 2005).
SANDGREN, A et al., “Tuberculosis drug resistance mutation database,” PLoS Med., 6:e2 (February 2009).
SANG, L et al., “Immunogenicity and protective efficacy against tuberculosis with DNA expressing ESAT6 protein,” Afr. J. Microbiol. Res., 6(2):431-435 (January 2012).
SAVINO, R et al., “In mesopore protein digestion: A new forthcoming strategy in proteomics,” Molecules, 16(7):5938-5962 (July 2011).
SCARPELLINI, P et al., “Selected pool of peptides from ESAT-6 and CFP-10 proteins for detection of Mycobacterium tuberculosis infection, ”J. Clin. Microbiol., 42(8):3469-3474 (August 2004).
SHEN, G H et al., “Rapid identification of the Mycobacterium tuberculosis complex by combining the ESAT-6/CFP-10 immunochromatographic assay and smear morphology,” J. Clin. Microbiol., 49(3):902-907 (March 2011).
SORENSEN, M H et al., “Expansion of the F127-templated mesostructure in aerosol-generated particles by using polypropylene glycol as a swelling agent,” Micropor. Mesopor. Mat., 113(1-3):1-13 (August 2008).
TORRES-CHAVOLLA, E, and ALOCILIA, E C, “Nanoparticle based DNA biosensor for tuberculosis detection using thermophilic helicase-dependent isothermal amplification,” Biosens. Bioelectron., 26(11):4614-4618 (July 2011).
TSAI, T T et al., “Paper-based tuberculosis diagnostic devices with colorimetric gold nanoparticles,” Sci. Technol. Adv. Mat., 14(4):044404 (August 2013).
VAN INGEN, J et al., “Region of difference 1 in nontuberculous Mycobacterium species adds a phylogenetic and taxonomical character,” J. Bacteriol., 191(18):5865-5867 (July 2009).
VINU, A et al., “Adsorption of lysozyme over mesoporous molecular sieves MCM-41 and SBA-15: influence of pH and aluminum incorporation,” J. Phys. Chem. B, 108(22):7323-7330 (May 2004).
WU, H-J et al., “Antibody-free detection of Mycobacterium tuberculosis antigen using customized nanotraps,” Anal. Chem., 86(4):1988-1996 (January 2014).
WU-HSIEH, B A et al., “Long-lived immune response to early secretory antigenic target 6 in individuals who had recovered from tuberculosis,” Clin. Infect. Dis., 33(8):1336-1340 (September 2001).

It should be understood that the examples and embodiments described herein are for illustrative purposes only and that various modifications or changes in light thereof will be suggested to persons skilled in the art and are to be included within the spirit and purview of this application and the scope of the appended claims.

All references, including publications, patent applications and patents, cited herein are hereby incorporated by reference to the same extent as if each reference was individually and specifically indicated to be incorporated by reference and was set forth in its entirety herein.

Recitation of ranges of values herein are merely intended to serve as a shorthand method of referring individually to each separate value falling within the range, unless otherwise indicated herein, and each separate value is incorporated into the specification as if it were individually recited herein.

The description herein of any aspect or embodiment of the invention using terms such as “comprising,” “having,” “including,” or “containing,” with reference to an element or elements is intended to provide support for a similar aspect or embodiment of the invention that “consists of,” “consists essentially of,” or “substantially comprises,” that particular element or elements, unless otherwise stated or clearly contradicted by context (e.g., a composition described herein as comprising a particular element should be understood as also describing a composition that contains and/or that includes that particular element, unless otherwise explicated stated, or clearly contradicted by context).

What is claimed is:

1. A method of identifying at least one pathogen-specific peptide or polypeptide from a biological sample, comprising contacting the sample with a nanoporous dual- or multi-layer silica film; and detecting the presence of the pathogen-specific peptide or polypeptide, or one or more proteolytic fragment(s) thereof.

2. The method of claim 1, wherein the pathogen-specific peptide or polypeptide is an M. tuberculosis-specific peptide or polypeptide.

3. The method of claim 2, wherein the M. tuberculosis-specific peptide or polypeptide comprises a contiguous amino acid sequence from an early secretory antigenic target protein (ESAT-6) or a culture filtrate protein 10 (CFP-10).

4. The method of claim 1, wherein the biological sample is obtained from a mammal.

5. The method of claim 1, wherein the biological sample comprises sputum, pleural effusion, cerebrospinal fluid, urine, serum, plasma, or whole blood.

6. The method of claim 1, wherein the at least one peptide or polypeptide within the biological sample is concentrated prior to contact with the nanoporous dual- or multi-layer silica film.

7. The method of claim 1, wherein the nanoporous dual- or multi-layer silica film comprises at least a first layer of silica film comprising a plurality of pores of substantially the same average diameter, into which the at least one pathogen-specific peptide or polypeptide is absorbed.

8. The method of claim 1, wherein the nanoporous dual- or multi-layer silica film comprises at least a first layer of silica film comprising a plurality of pores having an average diameter of about 3 to about 10 nm.

9. The method of claim 8, wherein the nanoporous dual- or multi-layer film comprises at least a first layer of silica film comprising a plurality of pores having an average diameter of about 6 to about 8 nm.

10. The method of claim 7, wherein the nanoporous dual- or multi-layer film comprises a second layer of silica film positioned upon the first layer, the second layer comprising a plurality of pores having an average diameter that is different from that of the pores of the first layer.

11. The method of claim 10, wherein the second layer of silica film contains a plurality of pores having a first average diameter that is larger than that of the plurality of pores in the first layer.

12. The method of claim 1, further comprising washing the nanoporous film after contacting the film with the biological sample.

13. The method of claim 1, further comprising digesting the sample containing the pathogen-specific peptide or polypeptide with a protease or a peptidase to produce one or more proteolytic fragment(s) of the pathogen-specific peptide or polypeptide.

14. The method of claim 13, wherein the protease is trypsin.

15. The method of claim 13, wherein proteolysis of the sample is performed on or within the nanoporous dual- or multi-layer silica film.

16. The method of claim 15, further comprising isolating the one or more proteolytic fragment(s) from the nanoporous dual- or multi-layer silica film with an elution buffer.

17. The method of claim 13, wherein the presence of the pathogen-specific peptide or polypeptide, or the one or more proteolytic fragment(s) thereof is detected by identifying at least one mass fingerprint of the peptide, the polypeptide, the proteolytic fragment(s), or a combination thereof, by mass spectrometry.

18. The method of claim 17, wherein the at least one mass fingerprint is detected by matrix-assisted laser desorption/ionization time-of-flight mass spectrometry (MALDI-TOF MS).

19. The method of claim 18, wherein the at least one mass fingerprint is identified at about 1895-1910 Da ([M+H]⁺) at about 2003-2005 Da ([M+H]⁺) at about 1900.9511 Da ([M+H]⁺) at about 1907.9246 Da ([M+H]⁺) at about 2003.9781 Da ([M+H]⁺) at about 1668.7170 Da ([M+H]⁺) at about 1593.7503 Da ([M+H]⁺) at about 1142.6276 Da ([M+H]⁺) at about 908.4584 Da ([M+H]⁺) or any combination thereof.

20. The method of claim 3, wherein the M. tuberculosis-specific peptide or polypeptide comprises an at least 8 contiguous amino acid sequence from any one of SEQ ID NO:1, SEQ ID NO:2, SEQ ID NO:3, SEQ ID NO:4, SEQ ID NO:5, SEQ ID NO:6, and SEQ ID NO:7.

21. The method of claim 20, wherein the M. tuberculosis-specific peptide or polypeptide comprises an at least 12 contiguous amino acid sequence from any one of SEQ ID NO:1, SEQ ID NO:2, SEQ ID NO:3, SEQ ID NO:4, SEQ ID NO:5, and SEQ ID NO:6.

22. The method of claim 21, wherein the M. tuberculosis-specific peptide or polypeptide comprises an at least 14 contiguous amino acid sequence from any one of SEQ ID NO:1, SEQ ID NO:2, SEQ ID NO:3, SEQ ID NO:4, and SEQ ID NO:5.

23. A method, comprising:
a) enriching at least one target protein or polypeptide from a sample by contacting the sample with a nanoporous dual- or multi-layer silica film under conditions to absorb the target protein or polypeptide to the film, and subsequently washing the nanoporous dual- or multi-layer silica film to remove extraneous material;
b) digesting the enriched target protein or polypeptide on the nanoporous dual- or multi-layer silica film to produce at least one digestion product comprising at least one proteolytic fragment thereof; and
c) detecting the presence of the at least one proteolytic fragment of the target protein or polypeptide.

24. The method of claim 23, wherein the target protein or polypeptide is specific to a pathogen associated with an infectious disease.

25. The method of claim 23, wherein the nanoporous dual- or multi-layer silica film comprises at least a first layer having a plurality of pores with an average pore diameter of about 3- to about 10-nm.

26. The method of claim 23, wherein the at least one target protein or polypeptide is an ESAT-6- or a CFP-10-specific protein or polypeptide.

27. The method of claim 23, wherein the detecting is performed using mass spectrometry.

28. The method of claim 24, wherein the infectious disease is tuberculosis.
